(19) United States
(12) Patent Application Publication
(10) Pub. No.: US 2016/0161495 A1
(43) Pub. Date: Jun. 9, 2016

(54) Method of Treatment

(71) Applicants: TETRALOGIC BIRINIPANT UK LTD, West Midlands (GB); HEALTH RESEARCH INC., Buffalo, NY (US)

(72) Inventors: C. Glenn Begley, Westlake Village, CA (US); Srinivas Chunduru, Phoenixville, PA (US); Mark McKinlay, Phoenixville, PA (US); Hans Minderman, Buffalo, NY (US); Yasuhiro Mitsuuchi, Phoenixville, PA (US); David Weng, Chevy Chase, MD (US)

(73) Assignee: Health Researc Inc., Buffalo, NY (US)

(21) Appl. No.: 14/908,262

(22) PCT Filed: Jul. 30, 2014
(86) PCT No.: PCT/US2014/048841

§ 371 Date: Jan. 28, 2016

Related U.S. Application Data
(60) Provisional application No. 61/859,898, filed on Jul. 30, 2013.

(51) Int. Cl. G01N 33/574; G06Q 30/02; A61K 38/05

(52) U.S. Cl. CPC G01N 33/57496; A61K 38/05; G06Q 30/0269; G01N 2333/4703

(57) ABSTRACT

A method of diagnosing and treating proliferative disorders in connection with the use of a Smac mimetic.

### CROSS-REFERENCE TO RELATED APPLICATION

This application claims priority to and the benefit of U.S. Provisional Application No. 61/859,898, entitled, “Method of Treatment” filed Jul. 30, 2013, the entire content of which are hereby incorporated by reference in its entirety.

### FIELD OF THE INVENTION

The present invention describes a method of excluding patients from treatment with a Smac mimetic or treatment with a Smac mimetic in combination with a chemotherapeutic agent or biological agent. The present invention is therefore useful in the treatment of cancer, autoimmune diseases, and other disorders for which treatment with a Smac mimetic is generally indicated.

### BACKGROUND OF THE INVENTION

### Apoptosis

Apoptosis (programmed cell death) plays a central role in the development and homeostasis of all multi-cellular organisms. Apoptosis can be initiated within a cell from an external factor such as a cytokine (extrinsic pathway) or via an intracellular event such as DNA damage (intrinsic pathway). Alterations in apoptotic pathways have been implicated in many types of human pathologies, including developmental disorders, cancer, autoimmune diseases, as well as neurodegenerative disorders. One mode of action of chemotherapeutic drugs is tumor cell death via apoptosis.

Apoptosis is conserved across species and executed primarily by activated caspases, a family of cysteine proteases with aspartate specificity in their substrates. These cysteine-containing aspartate-specific proteases (“caspases”) are produced in cells as catalytically inactive zymogens and are proteolytically processed to become active proteases during apoptosis. Once activated, effector caspases are responsible for proteolytic cleavage of a broad spectrum of cellular targets that ultimately lead to cell death. In normal surviving cells that have not received an apoptotic stimulus, most caspases remain inactive. If caspases are abnormally activated, their proteolytic activity can be inhibited by a family of evolutionarily conserved proteins called IAPs (inhibitors of apoptosis proteins).

Apoptotic mechanisms in cells are generally activated by either external stimuli (e.g., ligation of TNF superfamily receptors) or intrinsic stimuli (e.g., DNA damage). External signals such as ligation of TNF receptor family members (TNFR1, CD95, DR4, DR5) by their cognate ligands (TNFα, TRAIL, CD95L/Fas) or agonistic agents such as antibodies, result in assembly of protein complexes which serve to activate apical caspases such as caspase-8 or -10. For example, ligation of CD95 by CD95L/FasL results in assembly of the Death Inducing Signaling Complex (DISC) complex, which contains the Fas-associated Death Domain (FADD), caspase-8 and caspase-10. Intrinsic apoptotic signals converge on the mitochondria resulting in the depolarization of the outer mitochondrial membrane). This results in the release of proapoptotic factors, such as cytochrome C and the second mitochondria-derived activator of caspases (Smac). These factors result in both caspase activation and neutralization of IAP proteins enabling the completion of the apoptotic process.

### Inhibitor of Apoptosis Proteins (IAPs)

The IAP family of proteins suppresses apoptosis by preventing the activation of pro-caspases and/or inhibiting the enzymatic activity of mature caspases. Several distinct mammalian IAPs including XIAP, cIAP1, cIAP2, ML-IAP, NAIP (neuronal apoptosis inhibiting protein), ILP-2, Bruce, and survivin, have been identified, and they all exhibit anti-apoptotic activity in cell culture. IAPs were originally discovered in baculovirus-infected insect cells by their functional ability to substitute for p35 protein, an anti-apoptotic gene. IAPs have been described in organisms ranging from Drosophila to human, and are known to be overexpressed in many human cancers. Generally speaking, IAPs comprise one to three Baculovirus IAP repeat (BIR) domains, and most of them also possess a carboxyl-terminal RING finger motif. The BIR domain itself is a zinc binding domain of about 70 residues comprising 4 alpha-helices and 3 beta strands, with cysteine and histidine residues that coordinate the zinc ion. Specific BIR domains are believed to mediate the anti-apoptotic effect by inhibiting caspases and thus inhibiting apoptosis. XIAP, cIAP1 and cIAP2 are expressed ubiquitously in most adult and fetal tissues. Dysregulation of cIAPs has been correlated with poor prognosis in patients with various malignancies, and has been demonstrated to critically block activation of apoptotic molecular pathways. cIAPs have also been demonstrated to activate anti-apoptotic pathways through NF-κB. Overexpression of XIAP in tumor cells has been demonstrated to confer protection against a variety of pro-apoptotic stimuli and promotes resistance to chemotherapy. Consistent with this, a strong correlation between XIAP protein levels and survival has been demonstrated for patients with acute myelogenous leukemia. Down-regulation of XIAP expression by antisense oligonucleotides has been shown to sensitize tumor cells to death induced by a wide range of pro-apoptotic agents, both in vitro and in vivo.

### Smac/DIABLO

In normal cells signaled to undergo apoptosis, however, the IAP-mediated inhibitory effect must be removed, a process at least in part performed by a mitochondrial protein named Smac (second mitochondria-derived activator of caspases). Smac (or, DIABLO), is synthesized as a precursor molecule of 239 amino acids; the N-terminal 55 residues serve as the mitochondria targeting sequence that is removed after import. The mature form of Smac contains 184 amino acids and behaves as an oligomer in solution. Smac and various fragments thereof have been proposed for use as targets for identification of therapeutic agents.

Smac is synthesized in the cytoplasm with an N-terminal mitochondrial targeting sequence that is proteolytically removed during maturation to the mature polypeptide and is then targeted to the inter-membrane space of the mitochondria. At the time of apoptosis induction, Smac is released from mitochondria into the cytosol, together with cytochrome c, where it binds to IAPs, and enables caspase activation, therein eliminating the inhibitory effect of IAPs on apoptosis. Whereas cytochrome c induces multimerization of Apaf-1 to activate procaspase-9 and -3, Smac eliminates the inhibitory effect of multiple IAPs. Smac interacts with essentially all IAPs that have been examined to date including XIAP, cIAP1, cIAP2, ML-IAP, and survivin. Thus, Smac appears to be a master regulator of apoptosis in mammals.

It has also been shown that Smac promotes the enzymatic activation of mature caspases that are bound to XIAP. X-ray crystallography has shown that the first four amino acids Ala-Val-Pro-Ile (AVPI) of mature Smac bind to a portion of IAPs. This N-terminal sequence is essential for binding IAPs and blocking their anti-apoptotic effects.

In addition, IAPs also modulate signaling initiated by cell surface death receptors. Evidence implicating cIAP1 and cIAP2 in TNFα-mediated NF-κB activation originated from studies demonstrating that cIAPs can interact with TRAF2 which can bind to TNF receptors (Shu HB, 1996, Rothe M., 1995). An interesting feature of cIAP1 and cIAP2 is the presence of a C-terminal RING finger domain with E3 ubiquitin ligase activity. It has been demonstrated that cIAP1 and cIAP2 act as K63 E3 ubiquitin ligases for RIP1 in the TNFR1 signaling pathway (Bertrand et al., 2008). cIAP-mediated polyubiquination of RIP1 allows RIP1 to associate with the prosurvival TAB/TAK1 and IKK complex (IKKα/IKKβ/IKKγ) which facilitates activation of a downstream target, NF-κB, to promote cell survival and resistance to standard chemotherapies. NF-κB is a family of transcription factors that can be present in cells in an active state or in an inactive state. NF-κB, therefore, responds quickly to various inducers of NF-κB activity, e.g., reactive oxygen species, TNF-alpha, IL-1beta, bacterial lipopolysaccharides and receptor activator of NF-κB (RANK).

### Smac Mimetics

Smac mimetics, also known as IAP antagonists, are synthetic small molecules that mimic the structure and IAP antagonist activity of the four N-terminal amino acids of Smac, AVPI. When administered to animals suffering proliferative disorders, Smac mimetics antagonize IAPs, causing an increase in apoptosis among abnormally proliferating cells. Smac mimetic-induced degradation of cIAP1 inhibits TNF-mediated NF-κB activation.

Birinapant is a Smac mimetic that is described in U.S. Pat. No. 8,283,372 and referred to therein as Compound 15. It has the following structure:

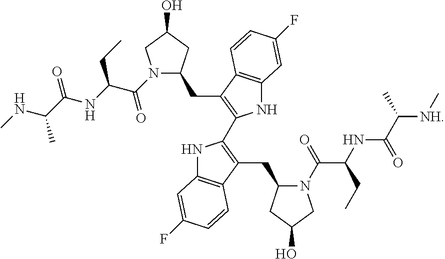

### BRIEF DESCRIPTION OF THE FIGURE

FIG. 1 is a block diagram showing a representative example logic device through which reviewing or analyzing data relating to the present invention can be achieved.

### SUMMARY OF THE INVENTION

In general, this invention provides practical and specific applications of the discovery that abnormally proliferating cells, e.g., cells in which the pro-apoptotic/pro-survival pathways are out of balance in favor of pro-survival, are unlikely to respond to treatment with a Smac mimetic (“SM”) if the basal level of NF-κB activation, as manifested, e.g., presence of phosphorylated NF-κB proteins in the cell nucleus, is low or non-existent. Such applications include, e.g., methods of treating patients based upon predicted resistance of cells, e.g., cancer cells or cells infected with a pathogenic microorganism, to treatment with a SM, alone, i.e., in monotherapy, or in combination with other therapies, e.g., co-administration with chemotherapeutic or biological agents. Stated another way, in certain embodiments, the invention relates to a method for excluding Smac mimetic therapy from patients who are unlikely to respond to such treatment.

A cell is sensitive to a SM, such as birinapant, if it undergoes apoptosis in response to the SM or if it is made more susceptible to apoptosis upon treatment with a chemotherapeutic agent (including without limitation biological therapies, e.g., cytokines), or radiation. Methods of the invention are useful for predicting which cells are unlikely to respond to treatment with a SM. The methods can be used either in laboratory or clinical settings.

Thus, in specific illustrative embodiments, the invention comprises, for example, a method of predicting the likelihood of resistance of abnormally proliferating cells to treatment with a SM that comprises assaying a sample of such cells for activation of the NF-κB signal transduction pathway, whereby if said pathway is not activated, then it is unlikely that the patient will respond to SM therapy.

In another embodiment, the invention comprises a method of predicting the likelihood of efficacy of treatment of a proliferative disorder with a SM that comprises:

(i) obtaining or having obtained a sample of abnormally proliferating cells associated with the disorder;
(ii) performing or having performed an assay to determine whether or not the NF-κB pathway is activated in such cells;
(iii) receiving a measure of activation of the NF-κB pathway in such cells;
wherein if the NF-κB pathway is not activated in such cells, then the likelihood of efficacy of the treatment is low or nil.

In another embodiment, the invention comprises a method of treating a patient suffering a proliferative disorder that comprises:

(i) obtaining or having obtained a sample of abnormally proliferating cells associated with the disorder;
(ii) performing or having performed an assay to determine whether or not the NF-κB pathway is activated in such cells;
(iii) receiving a measure of activation of the NF-κB pathway in such cells;
wherein if the NF-κB pathway is not activated in such cells, then treating the patient with a therapy other than SM therapy. In an alternative such embodiment, if the NF-κB pathway is activated, then the patient is treated with a SM, alone or in combination with an additional therapy.

In another embodiment, the invention comprises a method of stratifying patients suffering from cancer into at least two categories: (a) SM Therapy Candidates and (b) Non-SM Therapy Candidates, said method comprising:

(i) obtaining or having obtained a sample of abnormally proliferating cells associated with the disorder;
(ii) performing or having performed an assay to determine whether or not the NF-κB pathway is activated in such cells;
(iii) receiving a measure of activation of the NF-κB pathway in such cells;
wherein if the NF-κB pathway is activated in such cells from a given patient, then placing that patient in the SM Therapy Candidates category and treating the patient with a SM alone or in combination with an alternative therapy to SM therapy; and,
if the NF-κB pathway is not activated in such cells from a given patient, then placing that patient in the Non-SM Therapy Candidates category and treating the patient with an alternative therapy to SM therapy.

In another embodiment, the invention comprises a method of marketing a drug product comprising a SM for the treatment of a proliferative disorder, said method comprising informing patients, physicians or other healthcare providers, or insurers, that the SM is unlikely to be effective in treating patients whose abnormally proliferating cells have an undetectable or low basal level of NF-κB activation. Such embodiment can include, e.g., including in the prescribing information for a drug product comprising a SM: (1) information, e.g., data relating to the resistance of cells with low/undetectable levels of NF-κB to the drug; (2) advice, e.g., a recommendation or an instruction that the physician should or may choose to consider whether or not abnormally proliferating cells in a sample taken from the patient have low/undetectable levels of activated NF-κB prior to deciding to treat with a SM; (3) limiting the approved indication for the drug product to treatment with the drug only after the patient has been tested to determine if the patient's abnormally proliferating cells have low/undetectable levels of activated NF-κB, etc. . . .

In another embodiment, the invention comprises a computer system that comprises: (1) a computer including a computer processor; (2) a stored bit pattern encoding information regarding the state of activation of the NF-κB signal transduction pathway in abnormally proliferating cells from a patient.

In particular, illustrative embodiments, the human or non-human animal cells are from a sample taken from a patient, or they can be from a cell line. The cells may be any cells that are proliferating abnormally, e.g., tumor cells or cells that abnormally proliferate in an autoimmune disorder, and that are associated with the proliferative disorder to be treated, e.g., cancerous cells in a biopsy sample.

Thus, the invention in other illustrative embodiments also comprises a kit for the practice of the methods of the invention, such kit comprising, e.g., a means for detecting the activation of NF-κB in abnormally proliferating cells, such as described above and herein below.

In related aspects, the invention comprises a method of identifying a patient suffering from a proliferative disorder as a candidate or non-candidate for treatment with a SM that comprises obtaining a sample of diseased cells or tissue from the patient, determining the level of activation of the NF-κB pathway in such cells, and reporting information concerning the level of activation of the NF-κB pathway in such cells to a healthcare professional, e.g., the physician treating the patient, or directly to the patient. A decision on a treatment regimen is made in consideration of the level of NF-κB activation and, optionally, also in consideration of additional factors.

While this specification to a large degree describes the invention as it relates to proliferative disorders, e.g., cancers, it is to be understood that there are other medical indications for which treatment with a SM is or may be indicated. These can include, e.g., treatment of autoimmune disorders and also treatment of infection.

So, in further illustrative embodiments, the invention comprises a method of monitoring the response of a patient undergoing treatment with a SM, said method comprising monitoring levels of NF-κB activation in patient samples of target cells over time during a course of SM treatment, whereby decreasing levels of activated NF-κB in said cells is an indicator that the SM is having the desired pharmacologic effect. The target cells are those cells that are desirable to be eliminated, e.g., abnormally proliferating cells or infected cells.

In further illustrative embodiments, the invention comprises a method of treating a patient for whom SM therapy is indicated, said method comprising:

(a) internally administering a SM to the patient during at least one treatment cycle,
(b) monitoring the response of the patient to the SM during each treatment cycle by monitoring levels of activated NF-κB in patient samples of targeted cells over time, whereby decreasing levels of activated NF-κB in said cells is an indicator that the SM is having the desired pharmacologic effect, and
(c) (i) discontinuing the SM treatment following completion of the at least one treatment cycle if the level of activated NF-κB relative to the level of unactivated NF-κB in target cells does not decrease while the patient is being treated with the SM or
(c)(ii) continuing the SM treatment and the NF-κB monitoring for at least one additional treatment cycle following completion of the at least one treatment cycle if the level of activated NF-κB relative to the level of unactivated NF-κB in target cells decreases while the patient is being treated with the SM.

Related aspects include computer-based systems comprising means for receiving data concerning such pathway activation, optionally transiently or indefinitely storing such information, and directly or indirectly transmitting such information to such healthcare professional or patient. Such systems optionally include a program for analyzing and/or reporting NF-κB activation data.

The applications and uses of the methods described herein can produce one or more results useful as an aid to selection of an appropriate therapy for a patient suffering a proliferative disorder. In one embodiment, a method of diagnosing a disease comprises reviewing or analyzing data relating to activation of the NF-κB signal transduction pathway in a sample of abnormally proliferating cells removed from a patient. For example, in one embodiment the invention provides a method of predicting the likelihood of efficacy of treatment of a proliferative disorder with a SM by examining the biomarker of the invention, i.e., activation of NF-κB. A conclusion-based review or analysis of the data can be provided to the patient, a health care provider or a health care manager. In one embodiment, the conclusion is based on the review or analysis of data regarding a disease diagnosis. In another embodiment, the conclusion is based on the review or analysis of data regarding predicted drug efficacy/inefficacy. It is envisioned in yet another embodiment that providing a conclusion to a patient, a health care provider or a health care manager includes transmission of the data over a network. In a further embodiment, the data may be used to determine reimbursement for a treatment, e.g. whether an insurer will reimburse the healthcare provider for a particular drug. Accordingly, computer implemented systems and methods using the methods described herein are provided.

One aspect of the invention is a method comprising screening patient test samples for the biomarker of the invention, i.e., the level of NF-κB activation and optionally of one or more additional biomarkers, to produce data regarding the levels, collecting the data, providing the data to a patient, a health care provider or a health care manager for making a conclusion based on review or analysis of the data regarding a treatment method. In one embodiment the conclusion is provided to a patient, a health care provider or a health care manager includes transmission of the data over a network.

All aspects of the present invention specifically, but not exclusively, contemplate birinapant as the SM. So, for example, in illustrative embodiments, the invention comprises a method of treating a patient suffering from a cancer or autoimmune disease, or other proliferative disorder, or infection, by internally administering to the patient an effective amount of birinapant if the NF-κB pathway is activated in a sample of cells, e.g., a biopsy, taken from the patient.

### DETAILED DESCRIPTION OF THE INVENTION

In general, NF-κB is present in its inactive state in the cytoplasm and, upon activation, which includes a series of phosphorylation events, activated NF-κB translocates to the nucleus where it upregulates genes involved in development and proliferation, particularly T-cell development and proliferation. Thus, activation of NF-κB can be measured at any of several steps in the activation process.

The relative amounts of nuclear NF-κB can be determined by analysis with commerically available devices and/or systems that can differentiate and quantify the nuclear and cytoplasmic NF-κB, such as by a variety of digital microcopy-based imaging techniques. For example, preparations of cells can be fixed and analyzed using detectably labeled antibodies to NF-κB (such as to p65) and well known reagents to stain or otherwise identify the nucleus such that the nuclear and cytoplasmic NF-κB can be distinguished from one another. Suitable nuclear stains include but are not limited to 4′,6-diamidino-2-phenylindole (DAPI), Hoechst stains, Haematoxylin, Safranin, Carmine alum, and DRAQ5.

For example, one technique for detecting molecular translocation of NF-κB from the cytoplasm to the nucleus is imaging flow cytometry using, e.g., The ImageStream-X imaging flow cytometer (Amnis, Seattle, Wash.). Such device is described, e.g., in U.S. Pat. No. 7,522,758.

Among other methods that may be used, it is also possible to detect and measure p65, i.e., RelA (a member of the NF-κB family), using microscopy, e.g., by using anti-p65 labeled antibodies or anti-phosphorylated p65 labeled antibodies. The label is typically but not necessarily a fluorescent marker conjugated to the antibody.

A relative amount of NF-κB present in the nucleus can be represented by a similarity score, such as is described in US20110312016. In general, the smaller the similarity score, the less nuclear translocation of NF-κB and vice versa. More specifically, when imaging flow cytometry is employed, the similarity score is considered to be a log transformed Pearson's Correlation coefficient of the pixel by pixel intensity correlation between the cytoplasmic NF-κB and the nuclear stained (e.g., DRAQ5 image) NF-κB, which is calculated as a quantifiable parameter for the degree of NF-κB translocation to the nucleus. The similarity score (+ or −) is determined from the slope of the regression line while it takes its value from how well the individual pixel data points fit the regression line (Pearson correlation). A very low degree of nuclear translocation yields a highly negative similarity score while a very high degree of nuclear translocation yields a highly positive similarity score. It will therefore be recognized that, in one embodiment, a low degree of nuclear translocation can have anti-similar p65 and DRAQ5 images, while similar p65 and DRAQ5 images can yield a positive similarity score. Thus, in one embodiment, a negative similarity score obtained using an imaging flow cytometer system is indicative of resistance to SM therapy, while a highly positive similarity score is indicative of possible sensitivity to SM therapy. A standardized similarity score or ranges of similarity scores can accordingly be used as a control when performing the method of the invention.

The method of the invention can be repeated to monitor the NF-κB activation status of an individual over time. For example, the invention can be used to evaluate whether modifications of the therapy of an individual should be considered and/or implemented. The method of the invention can also be performed prior to initiation of SM therapy and compared to a biopsy sample(s) obtained from the individual after initiation of SM therapy.

In one embodiment, the method of the invention comprises communicating to a patient, physician or other healthcare provider, or an insurer, the result of determining an amount of activated NF-κB in cells or tissue from an individual, e.g., relative to the amount of unactivated, i.e., cytoplasmic, NF-κB.

In one embodiment, the method comprises fixing the result of determining the amount of nuclear NF-κB, e.g., relative to the amount of cytoplasmic NF-κB, in a tangible medium of expression, such as a digital medium, including but not limited to a compact disk, DVD, or any other memory device. Thus, the invention also provides a device or other tangible medium that contains a machine or human readable result from determining NF-κB activation levels. Imaging flow cytometery was initially used to show a time-dependent and dose-dependent reduction in phosphorylated RelA (a member of the NF-κB family) in the nucleus following treatment with birinapant of HL60 cells in which the NF-κB signal transduction pathway was previously activated by TNF-alpha. These results indicate that birinapant suppresses NF-κB activation in a time-dependent and dose-dependent manner.

More recently, imaging flow cytometry was used to detect translocation of NF-κB in cells from a subset of 12 patients diagnosed with MDS/AML in a phase I clinical study of birinapant. In five of the patients, in all of whom the basal level of nuclear NF-κB was undetectable or very low, the individual patient's AML blast counts did not decrease, i.e., the number of circulating myeloblasts did not decrease following birinapant treatment. Of the seven patients in whom translocation of NF-κB to the nucleus was detected prior to birinapant treatment, four showed decreased AML blast counts following treatment with birinapant while three did not. These results indicate that activated NF-κB is necessary for but not sufficient for a decrease in leukemic blast cells by treatment with a SM.

In this phase I clinical study, results from sixteen patients are available thus far (April, 2013). The patients studied were either refractory to multiple therapies or with relapsed disease following multiple therapies, and are typically elderly. The majority of patients have AML that had evolved from MDS. Ten patients received concurrent hydroxyurea during treatment with birinapant. Given the patient population, and that the study is still ongoing, the data and analyses are necessarily incomplete. However it is possible to draw some conclusions.

First, birinapant has been well-tolerated in these heavily pre-treated patients. This is true both as a single-agent, and in combination with hydroxyurea. Secondly, and as anticipated, there has been effective target suppression (cIAP1) with birinapant in leukemic cells. More important, is the inhibition of NF-κB (see below). Third, although there have been no formal objective responses, there is evidence of hematological effect in this phase 1 study. In five patients, and possibly a sixth, the decrease in circulating AML blast cells was attributable to birinapant. There has also been an increase in neutrophils although this may be a transient effect. Another patient with AML that had evolved from MDS, and who had progressive leukemic disease after prior cytotoxic regimens, had stable disease and no cumulative toxicities with 10 months of single-agent birinapant therapy.

Focusing specifically on patients who did not receive hydroxyurea, the emerging data suggests:

1) re-treatment with birinapant was associated with repeated response in terms of circulating AML blast counts. This implies that tumor cells remained responsive to birinapant; and,
2) resurgence of the circulating AML blast count appeared to be associated with breaks or delays in birinapant treatment.

Both conclusions also appear to be supported by results from patients who received hydroxyurea.

Based on the changes in circulating AML blast cells associated with birinapant, studies are ongoing to examine more dose-intense schedules of birinapant. Thus far it appears that birinapant administered twice per week may be more effective than weekly administration. This improvement in AML blast cell reduction with the more dose-intense administration of birinapant is supported by PK modeling data.

An additional component of this clinical study is to examine nuclear NF-κB in the peripheral AML blasts. Results of the NF-κB assay are only available on a subset of patients (n=12).

Interestingly, in five patients at baseline, the basal level of NF-κB in the nucleus was undetectable or very low. This suggests that alternative proliferative/survival pathways were active in these leukemic cells. Not surprisingly, these leukemic cells did not show any response to birinapant. Thus nuclear NF-κB might provide a future biomarker to exclude patients who are unlikely to respond to birinapant. Despite the lack of response in terms of leukemic blasts, these patients did show the transient increase in the absolute neutrophil count (in one patient, this increase was ˜5-fold).

In four patients, the inhibition of nuclear localization of NF-κB was associated with a decline in circulating AML blasts.

In three patients, there was no impact of birinapant on circulating AML blasts despite inhibition of nuclear localization of NF-κB. This is to be expected: there are multiple examples of targeted therapies that have the desired impact on intracellular signaling but without guaranteeing an impact on tumor growth.

While there is evidence of activity of birinapant in evolved MDS/AML, we anticipate greater activity will be seen in combination with agents that induce TNF. Single-agent birinapant activity is observed in many cancer and leukemia cell lines, however, the synergistic activity with agents that induce TNF is substantially more impressive. This enhanced activity is seen both in preclinical models and in clinical studies of solid tumors, and it is this activity that we seek to fully exploit in future clinical studies of MDS.

It is contemplated that the potential for NF-κB activation in response to treatment with agents such as, e.g., azacitidine, decitabine, irinotecan, etc., that activate NF-κB mediated pro-survival pathways can also be used as the biomarker, e.g., to exclude patients from SM monotherapy or SM combination therapy.

Although clinical data using birinapant in combination with TNF-inducing agents is still to be generated in AML/MDS, the compelling preclinical data, and the emerging clinical data provide a strong rationale for future studies.

In accordance with the present invention, activation of NF-κB can be used to identify abnormally proliferating cells, e.g., cancerous cells, that may be sensitive to the pro-apoptotic effects of a SM, e.g., birinapant. Cells in which NF-κB is not activated, or is activated only at low levels, are unlikely to respond to treatment with a SM.

Most cells will have baseline levels of active NF-κB, i.e., phosphorylated RelA in the nucleus and of inactive NF-κB, i.e., non-phosphorylated RelA in the cytoplasm. In broad terms, for purposes of this invention, “activated NF-κB” means that the amount of RelA in the nucleus is greater than the amount of RelA in the cytoplasm. This definition can be refined by establishing a range of normal nuclear NF-κB levels such that if a biopsy sample from a given patient shows nuclear NF-κB levels not greater than a range established for normal healthy donors, then the patient is unlikely to benefit from Smac mimetic treatment.

The treating physician, other healthcare worker, insurance provider, regulatory agency, or other person or entity involved in treatment of a cancer patient can establish a minimum degree of difference required to recommend treatment with a Smac mimetic. For example, in general, if the similarity coefficient for phosphorylated RelA and non-phosphorylated RelA is equal to or greater than 1, or about 1, then treatment with a Smac mimetic may be indicated; conversely, if it is less than 1, e.g., zero or negative, then treatment with a Smac mimetic may not be indicated.

NF-κB activation can vary between individuals, but also in the same individual based on numerous factors. For example, infection or allergies can increase NF-κB activation in a given individual. Therefore, the treating physician or other decision-maker may wish to measure NF-κB activation in cancerous cells multiple times over a period of time before deciding to initiate, or to continue, treatment with a Smac mimetic.

In some embodiments, the level of NF-κB activation below which SM therapy is not indicated may be set relative to the level of NF-κB activation in cells known to be sensitive to SM treatment. For example, the level of NF-κB activation that is equivalent to no NF-κB activation for purposes of predicting resistance to a SM may be set at 1% to 50%, e.g., 1%, 5%, 10%, 15%, 20%, 25%, 30%, 35%, 40%, 45%, or 50% of the level of NF-κB activation in abnormally proliferating cells shown to be sensitive to SM treatment.

Thus, NF-κB activation is a biomarker that can help guide a treating healthcare worker, e.g., a physician, in deciding whether or not to administer a pro-apoptotic agent to a patient suffering from a malignancy, or other disease state characterized, mediated, or exacerbated by abnormal cell proliferation. It is one of the factors that such healthcare provider can take into consideration when deciding what therapy to prescribe or, in the case of an insurer, which therapies to cover.

For example, if the targeted cells in a patient, e.g., cells in a biological sample of cells or tissue removed for biopsy, have NF-κB (RelA) in the nucleus, that marker is indicative of possible sensitivity to a SM (i.e., is indicative that the patient may respond to treatment). In that case, the healthcare provider, after considering other factors, such as, e.g., additional biomarkers, the patient's medical history, the type of cells, the stage of disease progression, etc., may decide that treatment with a SM is appropriate, as a primary or secondary therapy, alone or in combination with other drugs or other therapies.

Such biomarker can also be used to select patients for inclusion or exclusion in clinical studies designed to assess the efficacy of a pro-apoptotic agent. For example, cancer patients whose cancerous cells lack, or substantially lack, activated NF-κB are unlikely (or less likely) to respond to treatment with a SM and therefore may be excluded from enrollment or may be placed in a distinct arm of the study.

A Treatment Threshold can be set based on the level of NF-κB activation, i.e., the amount of NF-κB that has translocated to the nucleus. Such threshold corresponds to a likelihood of achieving efficacy of treatment with a SM. If the level of NF-κB activation is above the threshold, a decision may be made to treat the patient with the SM.

For example, a healthcare provider, perhaps in consultation with another(s), may decide that if the likelihood (or probability) that a given patient will respond to a given therapy is at least about 25%, then the patient will be treated with that therapy, but if the likelihood (or probability) that the patient will respond to the therapy is less than about 25%, then the patient will be treated with an alternative therapy. In this illustration, the Treatment Threshold is 25%. The Treatment Threshold can also be used as an inclusion/exclusion criterion for recruitment of subjects for clinical studies of pro-apoptotic agents.

Selection of a particular Treatment Threshold can be largely a subjective judgment based on numerous factors. Factors that can influence the selection of a Treatment Threshold include, e.g., the availability of alternative therapies, cost of treatment, and the risk of adverse events.

The Treatment Threshold can be set by each healthcare provider at a level that he or she is comfortable with, given other factors such as those already mentioned, or it can be pre-determined, e.g., by a drug regulatory agency. For example, a drug regulatory agency such as the U.S. Food and Drug Administration, based, e.g., on known adverse events and known alternative therapies, may approve a given pro-apoptotic agent for use in treatment of a specific proliferative disorder only in cases in which the Treatment Threshold for that drug and that disorder is a specified likelihood of response, e.g., 50% or approximately 50%.

A patient that “responds to treatment” is a patient for whom treatment with the drug is effective, i.e, a patient for whom treatment with a given pro-apoptic agent, alone or in combination with another chemo- or other therapy, results in stabilization or regression of the abnormal proliferation, e.g., as measured by tumor size, following treatment, such stabilization or regression occurring over a period of at least one month following treatment and the initial observation of stabilization or regression. In patients who are non-responders, the abnormal cell proliferation progresses or becomes stabilized or enters regression for a short duration, i.e., for less than a month following treatment and an initial observation of stabilization or regression.

In general, there is a direct relationship between absence of activated NF-κB in the nucleus and resistance to SM therapy.

It will be understood that cell or tissue samples from patients with a proliferative disorder will likely contain both abnormally proliferating cells, i.e., tumor or cancer cells, as well as healthy cells, e.g., stroma. Activation of NF-κB can be normalized against tumor content per sample. In this way, the concentration of abnormally proliferating cells in a given sample, i.e., the percentage of all cells in the sample that are abnormally proliferating cells, can be taken into account when determining the level of NF-κB activation in the patient's cancerous cells.

This invention also contemplates indirect measurement of activated/unactivated NF-κB in target cells such as by measuring activated/unactivated NF-κB in normal cells or in both normal and target cells, effectively using normal cells as a surrogate for the target cells. The activated NF-κB biomarker of the present invention can also be used in combination with other biomarkers of responsiveness or of safety.

A physician may prescribe that an activated NF-κB assay be carried out prior to administering a SM. Such test may be carried out by the physician but is more likely to be carried out by a diagnostic technician, such as a technician employed in a diagnostic laboratory. In this case, the physician may obtain a sample from a patient (or may request another person to obtain such sample) and then have the sample analyzed by a diagnostic laboratory. The physician then would receive from the diagnostic laboratory a report describing the patient's level of NF-κB activation. For example, such report may state that the extent of NF-κB activation in the sample indicates that a SM is unlikely to be effective. In any case, the report will indicate or be used by the physician to determine a likelihood of efficacy/inefficacy and the physician may then decide to treat or not to treat the patient based on the report, perhaps after also considering additional factors such as discussed above.

Any cell type can be assayed for NF-κB activation. The cells can be primary cells (e.g., cells of a biopsy obtained from a patient) or from cell lines. This invention does not require practice on the human or animal body. Of particular interest are cells which proliferate abnormally, including cells which proliferate pathologically and which are associated with the proliferative disorder that a patient is suffering from, i.e., cells that cause or lead to disease symptoms. Smac mimetics include, without limitation, the IAP antagonists disclosed in U.S. Pat. No. 7,517,906; U.S. Pat. No. 7,419,975; U.S. Pat. No. 7,589,118; U.S. Pat. No. 7,932,382; U.S. Pat. No. 7,345,081; U.S. Pat. No. 7,244,851; U.S. Pat. No. 7,674,787; U.S. Pat. No. 7,772,177; U.S. Pat. No. 7,989,441; U.S. Pat. No. 8,163,792; U.S. Pat. No. 8,278,293; US20100324083; US20100056467; US20090069294; US20110065726; US20110206690; US20130172264, WO2011098904.

The compounds disclosed therein, and Smac mimetics generally, have the structure:

[P1-P2-P3-P4] (Formula I)

or

[P1-P2-P3-P4]-L-[P1′-P2′-P3′-P4′] (Formula II)

wherein P1-P2-P3- and P1′-P2′-P3′- correspond to peptide replacements, i.e., peptidomimetics, of the N-terminal Ala-Val-Pro-tripeptide of mature Smac and P4 and P4′ correspond to amino acid replacements of the fourth N-terminal amino acid, Phe, Tyr, Ile, or Val, and L is a linking group or bond covalently linking [P1-P2-P3-P4] to [P1′-P2′-P3′-P4′].

For example, without limitation, a Smac mimetic may reside in the following genus of compounds of Formula II:

P1 and P1′ are NHR¹—CHR²—C(O)—;

### P2 and P2′ are —NH—CHR³—C(O)—;

P3 and P3′ are pyrrolidine, pyrrolidine fused to a cycloalkyl, or pyrrolidine fused to a heterocycloalkyl having a—N— heteroatom, optionally substituted in each case, and wherein the pyrrolidine of P3/P3′ is bound to P2/P2′ by an amide bond;
P4 and P4′ are -M-Qₚ-R⁷.
The variable substituents can be, for example:

### R¹: —H or —CH3;

### R²: —CH3, —CH2CH3 or —CH2OH;

R³: C2-6 alkyl, C2-6 alkoxy, C3-C6 cycloalkyl or heterocycloalkyl, or C6-C8 aryl or heteroaryl, optionally substituted in each case;
M: a covalent bond, C1-6 alkylene, substituted C1-C6 alkylene such as but not limited to —C(O)—;
Q: a covalent bond, C1-6 alkylene, substituted C1-C6 alkylene, —O— or —NR⁸—,

### P: 0 or 1;

R⁷: cycloalkyl, cycloalkylaryl, alkylaryl, alkylheteroaryl, aryl or heteroaryl, optionally substituted in each case;
R⁸: —H or C1-6 alkyl.

L is a linking group or bond covalently linking [P1-P2-P3-P4] to [P1′-P2′-P3′-P4]. “Alkyl” (monovalent) and “alkylene” (divalent) when alone or as part of another term (e.g., alkoxy) mean branched or unbranched, saturated aliphatic hydrocarbon group, having up to 12 carbon atoms unless otherwise specified. Examples of particular alkyl groups include, but are not limited to, methyl, ethyl, n-propyl, isopropyl, n-butyl, iso-butyl, sec-butyl, tert-butyl, n-pentyl, 2-methylbutyl, 2,2-dimethylpropyl, n-hexyl, 2-methylpentyl, 2,2-dimethylbutyl, n-heptyl, 3-heptyl, 2-methylhexyl, and the like. The term, “lower,” when used to modify alkyl, alkenyl, etc., means 1 to 4 carbon atoms, branched or linear so that, e.g., the terms “lower alkyl”, “C₁-C₄ alkyl” and “alkyl of 1 to 4 carbon atoms” are synonymous and used interchangeably to mean methyl, ethyl, 1-propyl, isopropyl, 1-butyl, sec-butyl or t-butyl. Examples of alkylene groups include, but are not limited to, methylene, ethylene, n-propylene, n-butylene and 2-methyl-butylene.

The term substituted alkyl refers to alkyl moieties having substituents replacing one or more hydrogens on one or more (often no more than four) carbon atoms of the hydrocarbon backbone. Such substituents are independently selected from the group consisting of: a halogen (e.g., I, Br, Cl, or F, particularly fluoro(F)), hydroxy, amino, cyano, mercapto, alkoxy (such as a C₁-C₆ alkoxy, or a lower (C₁-C₄) alkoxy, e.g., methoxy or ethoxy to yield an alkoxyalkyl), aryloxy (such as phenoxy to yield an aryloxyalkyl), nitro, oxo (e.g., to form a carbonyl), carboxyl (which is actually the combination of an oxo and hydroxy substituent on a single carbon atom), carbamoyl (an aminocarbonyl such as NR₂C(O)—, which is the substitution of an oxo and an amino on a single carbon atom), cycloalkyl (e.g., a cycloalkylalkyl), aryl (resulting for example in aralkyls such as benzyl or phenylethyl), heterocyclylalkyl (e.g., heterocycloalkylalkyl), heteroaryl (e.g., heteroarylalkyl), alkylsulfonyl (including lower alkylsulfonyl such as methylsulfonyl), arylsulfonyl (such as phenylsulfonyl), and —OCF₃ (which is a halogen substituted alkoxy). The invention further contemplates that several of these alkyl substituents, including specifically alkoxy, cycloalkyl, aryl, heterocyclyalkyl and heteroaryl, are optionally further substituted as defined in connection with each of their respective definitions provided below. In addition, certain alkyl substituent moieties result from a combination of such substitutions on a single carbon atom. For example, an ester moiety, e.g., an alkoxycarbonyl such as methoxycarbonyl, or tert-butoxycarbonyl (Boc) results from such substitution. In particular, methoxycarbonyl and Boc are substituted alkyls that result from the substitution on a methyl group (—CH₃) of both an oxo (═O) and an unsubstituted alkoxy, e.g., a methoxy (CH₃—O) or a tert-butoxy ((CH₃)₃C—O—), respectively replacing the three hydrogens. Similarly, an amide moiety, e.g., an alkylaminocarbonyl, such as dimethlyaminocarbonyl or methylaminocarbonyl, is a substituted alkyl that results from the substitution on a methyl group (—CH₃) of both an oxo (═O) and a mono-unsubstitutedalkylamino or, diunsubstitutedalkylamino, e.g., dimethylamino (—N—(CH₃)₂), or methylamino (—NH—(CH₃)) replacing the three hydrogens (similarly an arylaminocarbonyl such as diphenylaminocarbonyl is a substituted alkyl that results from the substitution on a methyl group (—CH₃) of both an oxo (═O) and a mono-unsubstitutedaryl(phenyl)amino). Exemplary substituted alkyl groups further include cyanomethyl, nitromethyl, hydroxyalkyls such as hydroxymethyl, trityloxymethyl, propionyloxymethyl, aminoalkyls such as aminomethyl, carboxylalkyls such as carboxymethyl, carboxyethyl, carboxypropyl, 2,3-dichloropentyl, 3-hydroxy-5-carboxyhexyl, acetyl (e.g., an alkanoyl, where in the case of acetyl the two hydrogen atoms on the —CH₂ portion of an ethyl group are replaced by an oxo (═O)), 2-aminopropyl, pentachlorobutyl, trifluoromethyl, methoxyethyl, 3-hydroxypentyl, 4-chlorobutyl, 1,2-dimethyl-propyl, pentafluoroethyl, alkyloxycarbonylmethyl, allyloxycarbonylaminomethyl, carbamoyloxymethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro(n-butyl), 2-amino(iso-propyl), cycloalkylcarbonyl (e.g., cuclopropylcarbonyl) and 2-carbamoyloxyethyl. Particular substituted alkyls are substituted methyl groups. Examples of substituted methyl group include groups such as hydroxymethyl, protected hydroxymethyl (e.g., tetrahydropyranyl-oxymethyl), acetoxymethyl, carbamoyloxymethyl, trifluoromethyl, chloromethyl, carboxymethyl, carboxyl (where the three hydrogen atoms on the methyl are replaced, two of the hydrogens are replaced by an oxo (═O) and the other hydrogen is replaced by a hydroxy (—OH)), tert-butoxycarbonyl (where the three hydrogen atoms on the methyl are replaced, two of the hydrogens are replaced by an oxo (═O) and the other hydrogen is replaced by a tert-butoxy (—O—C(CH₃)₃), bromomethyl and iodomethyl. When the specification and especially the claims refer to a particular substuituent for an alkyl, that substituent can potentially occupy one or more of the substitutable positions on the allkyl. For example, reciting that an alkyl has a fluoro substituent, would embrace mono-, di-, and possibly a higher degree of substitution on the alkyl moiety. The term substituted alkylene refers to alkylene moieties having substituents replacing one or more hydrogens on one or more (often no more than four) carbon atoms of the hydrocarbon backbone where the alkylene is similarly substituted with groups as set forth above for alkyl. Alkoxy is —O-alkyl. A substituted alkoxy is —O-substituted alkyl, where the alkoxy is similarly substituted with groups as set forth above for alkyl. One substituted alkoxy is acetoxy where two of the hydrogens in ethoxy (e.g., —O—CH₂—CH₃) are replaced by an oxo, (═O) to yield —O—C(O)—CH₃; another is an aralkoxy where one of the hydrogens in the alkoxy is replaced by an aryl, such as benzyloxy, and another is a carbamate where two of the hydrogens on methoxy (e.g., —O—CH₃) are replaced by oxo (═O) and the other hydrogen is replaced by an amino (e.g., —NH₂, —NHR or —NRR) to yield, for example, —O—C(O)—NH₂. A lower alkoxy is —O-lower alkyl.

“Alkenyl” (monovalent) and “alkenylene” (divalent) when alone or as part of another term mean an unsaturated hydrocarbon group containing at least one carbon-carbon double bond, typically 1 or 2 carbon-carbon double bonds, which may be linear or branched and which have at least 2 and up to 12 carbon atoms unless otherwise specified. Representative alkenyl groups include, by way of example, vinyl, allyl, isopropenyl, but-2-enyl, n-pent-2-enyl, and n-hex-2-enyl. The terms substituted alkenyl and substituted alkenylene refer to alkenyl and alkenylene moieties having substituents replacing one or more hydrogens on one or more (often no more than four) carbon atoms of the hydrocarbon backbone. Such substituents are independently selected from the group consisting of: halo (e.g., I, Br, Cl, F), hydroxy, amino, cyano, alkoxy (such as C₁-C₆ alkoxy), aryloxy (such as phenoxy), nitro, mercapto, carboxyl, oxo, carbamoyl, cycloalkyl, aryl, heterocyclyl, heteroaryl, alkylsulfonyl, arylsulfonyl and —OCF₃.

“Alkynyl” means a monovalent unsaturated hydrocarbon group containing at least one carbon-carbon triple bond, typically 1 carbon-carbon triple bond, which may be linear or branched and which have at least 2 and up to 12 carbon atoms unless otherwise specified. Representative alkynyl groups include, by way of example, ethynyl, propargyl, and but-2-ynyl.

“Cycloalkyl” when alone or as part of another term means a saturated or partially unsaturated cyclic aliphatic hydrocarbon group (carbocycle group), having 3 to 8 carbon atoms unless otherwise specified, such as cyclopropyl, cyclobutyl, cyclopentyl and cyclohexyl, and further includes polycyclic, including fused cycloalkyls such as 1,2,3,4-tetrahydonaphthalenyls (1,2,3,4-tetrahydonaphthalen-1-yl, and 1,2,3,4-tetrahydonaphthalen-2-yl), indanyls (indan-lyl, and indan-2-yl), isoindenyls (isoinden-1-yl, isoinden-2-yl, and isoinden-3-yl) and indenyls (inden-1-yl, inden-2-yl and inden-3-yl). A lower cycloalkyl has from 3 to 6 carbon atoms and includes cyclopropyl, cyclobutyl, cyclopentyl and cyclohexyl.

The term substituted cycloalkyl refers to cycloalkyl moieties having substituents replacing one or more hydrogens on one or more (often no more than four) carbon atoms of the hydrocarbon backbone. Such substituents are independently selected from the group consisting of: halo (e.g., I, Br, Cl, F), hydroxy, amino, cyano, alkoxy (such as C₁-C₆ alkoxy), substituted alkoxy, aryloxy (such as phenoxy), nitro, mercapto, carboxyl, oxo, carbamoyl, alkyl, substituted alkyls such as trifluoromethyl, aryl, substituted aryls, heterocyclyl, heteroaryl, alkylsulfonyl, arylsulfonyl and —OCF₃. When the specification and especially the claims refer to a particular substuituent for a cycloalkyl, that substituent can potentially occupy one or more of the substitutable positions on the cycloalkyl. For example, reciting that a cycloalkyl has a fluoro substituent, would embrace mono-, di-, and a higher degree of substitution on the cycloalkyl moiety. Examples of cycloalkyls include cyclopropy, cyclobutyl, cyclopentyl, cyclohexyl, tetrahydronaphthyl and indanyl.

“Aryl” when used alone or as part of another term means an aromatic carbocyclic group whether or not fused having the number of carbon atoms designated, or if no number is designated, from 6 up to 14 carbon atoms. Particular aryl groups include phenyl, naphthyl, biphenyl, phenanthrenyl, naphthacenyl, indolyl, and the like (see e.g. Lang's Handbook of Chemistry (Dean, J. A., ed) 13^{th} ed. Table 7-2 [1985]).

The term substituted aryl refers to aryl moieties having substituents replacing one or more hydrogens on one or more (usually no more than six) carbon atoms of the aromatic hydrocarbon core. Such substituents are independently selected from the group consisting of: halo (e.g., I, Br, Cl, F), hydroxy, amino, cyano, alkoxy (such as C₁-C₆ alkoxy and particularly lower alkoxy), substituted alkoxy, aryloxy (such as phenoxy), nitro, mercapto, carboxyl, carbamoyl, alkyl, substituted alkyl (such as trifluoromethyl), aryl, —OCF₃, alkylsulfonyl (including lower alkylsulfonyl), arylsulfonyl, heterocyclyl and heteroaryl. Examples of such substituted phenyls include but are not limited to a mono- or di (halo) phenyl group such as 2-chlorophenyl, 2-bromophenyl, 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl; 3-fluorophenyl, 4-fluorophenyl, a mono- or di (hydroxy) phenyl group such as 4-hydroxyphenyl, 3-hydroxyphenyl, 2,4-dihydroxyphenyl, the protected-hydroxy derivatives thereof; a nitrophenyl group such as 3- or 4-nitrophenyl; a cyanophenyl group, for example, 4-cyanophenyl; a mono- or di (lower alkyl) phenyl group such as 4-methylphenyl, 2,4-dimethylphenyl, 2-methylphenyl, 4-(iso-propyl) phenyl, 4-ethylphenyl, 3-(n-propyl)phenyl; a mono or di (alkoxy) phenyl group, for example, 3,4-dimethoxyphenyl, 3-methoxy-4-benzyloxyphenyl, 3-methoxy-4-(1-chloromethyl)benzyloxy-phenyl, 3-ethoxyphenyl, 4-(isopropoxy)phenyl, 4-(t-butoxy)phenyl, 3-ethoxy-4-methoxyphenyl; 3- or 4-trifluoromethylphenyl; a mono- or dicarboxyphenyl or (protected carboxy) phenyl group such 4-carboxyphenyl; a mono- or di (hydroxymethyl) phenyl or (protected hydroxymethyl) phenyl such as 3-(protected hydroxymethyl) phenyl or 3,4-di (hydroxymethyl) phenyl; a mono- or di (aminomethyl) phenyl or (protected aminomethyl) phenyl such as 2-(aminomethyl) phenyl or 2, 4-(protected aminomethyl) phenyl; or a mono- or di (N-(methylsulfonylamino)) phenyl such as 3-(N-methylsulfonylamino) phenyl. Also, the substituents, such as in a disubstituted phenyl groups, can be the same or different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, as well as for trisubstituted phenyl groups where the substituents are different, as for example 3-methoxy-4-benzyloxy-6-methyl sulfonylamino, 3-methoxy-4-benzyloxy-6-phenyl sulfonylamino, and tetrasubstituted phenyl groups where the substituents are different such as 3-methoxy-4-benzyloxy-5-methyl-6-phenyl sulfonylamino. Particular substituted phenyl groups are 2-chlorophenyl, 2-aminophenyl, 2-bromophenyl, 3-methoxyphenyl, 3-ethoxy-phenyl, 4-benzyloxyphenyl, 4-methoxyphenyl, 3-ethoxy-4-benzyloxyphenyl, 3,4-diethoxyphenyl, 3-methoxy-4-benzyloxyphenyl, 3-methoxy-4-(1-chloromethyl)benzyloxy-phenyl, 3-methoxy-4-(1-chloromethyl)benzyloxy-6-methyl sulfonyl aminophenyl groups. When the specification and especially the claims refer to a particular substuituent for an aryl, that substituent can potentially occupy one or more of the substitutable positions on the aryl. For example, reciting that an aryl has a fluoro substituent, would embrace mono-, di-, tri, tetra and a higher degree of substitution on the aryl moiety. Fused aryl rings may also be substituted with the substituents specified herein, for example with 1, 2 or 3 substituents, in the same manner as substituted alkyl groups. The terms aryl and substituted aryl do not include moieties in which an aromatic ring is fused to a saturated or partially unsaturated aliphatic ring.

“Heterocyclic group”, “heterocyclic”, “heterocycle”, “heterocyclyl”, “heterocycloalkyl” or “heterocyclo” alone and when used as a moiety in a complex group, are used interchangeably and refer to any mono-, bi-, or tricyclic, saturated or unsaturated, non-aromatic hetero-atom-containing ring system having the number of atoms designated, or if no number is specifically designated then from 5 to about 14 atoms, where the ring atoms are carbon and at least one heteroatom and usually not more than four heteroatoms (i.e., nitrogen, sulfur or oxygen). Included in the definition are any bicyclic groups where any of the above heterocyclic rings are fused to an aromatic ring (i.e., an aryl (e.g., benzene) or a heteroaryl ring). In a particular embodiment the group incorporates 1 to 4 heteroatoms. Typically, a 5-membered ring has 0 to 1 double bonds and a 6- or 7-membered ring has 0 to 2 double bonds and the nitrogen or sulfur heteroatoms may optionally be oxidized (e.g. SO, SO₂), and any nitrogen heteroatom may optionally be quaternized. Particular unsubstituted non-aromatic heterocycles include morpholinyl (morpholino), pyrrolidinyls, oxiranyl, indolinyls, 2,3-dihydoindolyl, isoindolinyls, 2,3-dihydoisoindolyl, tetrahydroquinolinyls, tetrahydroisoquinolinyls, oxetanyl, tetrahydrofuranyls, 2,3-dihydrofuranyl, 2H-pyranyls, tetrahydropyranyls, aziridinyls, azetidinyls, 1-methyl-2-pyrrolyl, piperazinyls and piperidinyls.

The term substituted heterocyclo refers to heterocyclo moieties having substituents replacing one or more hydrogens on one or more (usually no more than six) atoms of the heterocyclo backbone. Such substituents are independently selected from the group consisting of: halo (e.g., I, Br, Cl, F), hydroxy, amino, cyano, alkoxy (such as C₁-C₆ alkoxy), substituted alkoxy, aryloxy (such as phenoxy), nitro, carboxyl, oxo, carbamoyl, alkyl, substituted alkyl (such as trifluoromethyl), —OCF₃, aryl, substituted aryl, alkylsulfonyl (including lower alkylsulfonyl), and arylsulfonyl. When the specification and especially the claims refer to a particular substuituent for a heterocycloalkyl, that substituent can potentially occupy one or more of the substitutable positions on the heterocycloalkyl. For example, reciting that a heterocycloalkyl has a fluoro substituent, would embrace mono-, di-, tri, tetra and a higher degree of substitution on the heterocycloalkyl moiety.

“Heteroaryl” alone and when used as a moiety in a complex group refers to any mono-, bi-, or tricyclic aromatic ring system having the number of atoms designated, or if no number is specifically designated then at least one ring is a 5-, 6- or 7-membered ring and the total number of atoms is from 5 to about 14 and containing from one to four heteroatoms selected from the group consisting of nitrogen, oxygen, and sulfur (Lang's Handbook of Chemistry, supra). Included in the definition are any bicyclic groups where any of the above heteroaryl rings are fused to a benzene ring. The following ring systems are examples of the heteroaryl groups denoted by the term “heteroaryl”: thienyls (alternatively called thiophenyl), furyls, imidazolyls, pyrazolyls, thiazolyls, isothiazolyls, oxazolyls, isoxazolyls, triazolyls, thiadiazolyls, oxadiazolyls, tetrazolyls, thiatriazolyls, oxatriazolyls, pyridyls, pyrimidinyls (e.g., pyrimidin-2-yl), pyrazinyls, pyridazinyls, thiazinyls, oxazinyls, triazinyls, thiadiazinyls, oxadiazinyls, dithiazinyls, dioxazinyls, oxathiazinyls, tetrazinyls, thiatriazinyls, oxatriazinyls, dithiadiazinyls, imidazolinyls, dihydropyrimidyls, tetrahydropyrimidyls, tetrazolo[1,5-b)]pyridazinyl and purinyls, as well as benzo-fused derivatives, for example benzoxazolyls, benzofuryls, benzothienyls, benzothiazolyls, benzothiadiazolyl, benzotriazolyls, benzoimidazolyls, isoindolyls, indazolyls, indolizinyls, indolyls, naphthyridines, pyridopyrimidines, phthalazinyls, quinolyls, isoquinolyls and quinazolinyls.

The term substituted heteroaryl refers to heteroaryl moieties (such as those identified above) having substituents replacing one or more hydrogens on one or more (usually no more than six) atoms of the heteroaryl backbone. Such substituents are independently selected from the group consisting of: halo (e.g., I, Br, Cl, F), hydroxy, amino, cyano, alkoxy (such as C₁-C₆ alkoxy), aryloxy (such as phenoxy), nitro, mercapto, carboxyl, carbamoyl, alkyl, substituted alkyl (such as trifluoromethyl), —OCF₃, aryl, substituted aryl, alkylsulfonyl (including lower alkylsulfonyl), and arylsulfonyl. When the specification and especially the claims refer to a particular substuituent for a heteroaryl, that substituent can potentially occupy one or more of the substitutable positions on the heteroaryl. For example, reciting that a heteroaryl has a fluoro substituent, would embrace mono-, di-, tri, tetra and a higher degree of substitution on the heteroaryl moiety.

Particular “heteroaryls” (including “substituted heteroaryls”) include; 1H-pyrrolo[2,3-b]pyridine, 1,3-thiazol-2-yl, 4-(carboxymethyl)-5-methyl-1,3-thiazol-2-yl, 1,2,4-thiadiazol-5-yl, 3-methyl-1,2,4-thiadiazol-5-yl, 1,3,4-triazol-5-yl, 2-methyl-1,3,4-triazol-5-yl, 2-hydroxy-1,3,4-triazol-5-yl, 2-carboxy-4-methyl-1,3,4-triazol-5-yl, 1,3-oxazol-2-yl, 1,3,4-oxadiazol-5-yl, 2-methyl-1,3,4-oxadiazol-5-yl, 2-(hydroxymethyl)-1,3,4-oxadiazol-5-yl, 1,2,4-oxadiazol-5-yl, 1,3,4-thiadiazol-5-yl, 2-thiol-1,3,4-thiadiazol-5-yl, 2-(methylthio)-1,3,4-thiadiazol-5-yl, 2-amino-1,3,4-thiadiazol-5-yl, 1H-tetrazol-5-yl, 1-methyl-1H-tetrazol-5-yl, 1-(1-(dimethylamino) eth-2-yl)-1H-tetrazol-5-yl, 1-(carboxymethyl)-1H-tetrazol-5-yl, 1-(methylsulfonic acid)-1H-tetrazol-5-yl, 2-methyl-1H-tetrazol-5-yl, 1,2,3-triazol-5-yl, 1-methyl-1,2,3-triazol-5-yl, 2-methyl-1,2,3-triazol-5-yl, 4-methyl-1,2,3-triazol-5-yl, pyrid-2-yl N-oxide, 6-methoxy-2-(n-oxide)-pyridaz-3-yl, 6-hydroxypyridaz-3-yl, 1-methylpyrid-2-yl, 1-methylpyrid-4-yl, 2-hydroxypyrimid-4-yl, 1,4,5,6-tetrahydro-5,6-dioxo-4-methyl-as-triazin-3-yl, 1, 4,5,6-tetrahydro-4-(formylmethyl)-5,6-dioxo-as-triazin-3-yl, 2,5-dihydro-5-oxo-6-hydroxy-astriazin-3-yl, 2,5-dihydro-5-oxo-6-hydroxy-as-triazin-3-yl, 2,5-dihydro-5-oxo-6-hydroxy-2-methyl-astriazin-3-yl, 2,5-dihydro-5-oxo-6-hydroxy-2-methyl-as-triazin-3-yl, 2,5-dihydro-5-oxo-6-methoxy-2-methyl-as-triazin-3-yl, 2,5-dihydro-5-oxo-as-triazin-3-yl, 2,5-dihydro-5-oxo-2-methyl-as-triazin-3-yl, 2,5-dihydro-5-oxo-2,6-dimethyl-as-triazin-3-yl, tetrazolo[1,5-b]pyridazin-6-yl, 8-aminotetrazolo[1,5-b]-pyridazin-6-yl, quinol-2-yl, quinol-3-yl, quinol-4-yl, quinol-5-yl, quinol-6-yl, quinol-8-yl, 2-methyl-quinol-4-yl, 6-fluoro-quinol-4-yl, 2-methyl, 8-fluoro-quinol-4-yl, isoquinol-5-yl, isoquinol-8-yl, isoquinol-1-yl, and quinazolin-4-yl. An alternative group of “heteroaryl” includes: 5-methyl-2-phenyl-2H-pyrazol-3-yl, 4-(carboxymethyl)-5-methyl-1,3-thiazol-2-yl, 1,3,4-triazol-5-yl, 2-methyl-1,3,4-triazol-5-yl, 1H-tetrazol-5-yl, 1-methyl-1H-tetrazol-5-yl, 1-(1-(dimethylamino) eth-2-yl)-1H-tetrazol-5-yl, 1-(carboxymethyl)-1H-tetrazol-5-yl, 1-(methylsulfonic acid)-1H-tetrazol-5-yl, 1,2,3-triazol-5-yl, 1,4,5,6-tetrahydro-5,6-dioxo-4-methyl-as-triazin-3-yl, 1, 4,5,6-tetrahydro-4-(2-formylmethyl)-5,6-dioxo-as-triazin-3-yl, 2,5-dihydro-5-oxo-6-hydroxy-2-methyl-as-triazin-3-yl, 2,5-dihydro-5-oxo-6-hydroxy-2-methyl-as-triazin-3-yl, tetrazolo[1,5-b]pyridazin-6-yl, and 8-aminotetrazolo[1,5-b]pyridazin-6-yl. L is a linking group or a bond covalently linking one monomer, [P1-P2-P3-P4] to the other monomer, [P1′-P2′-P3′-P4′]. Commonly, -L-links P2 to P2′ position such as at R3 or P4 to P4′ such as at M, G, Q, or R⁷, or both P2 to P2′ and P4 to P4′. L, therefore, can be a single or double covalent bond or a contiguous chain, branched or unbranched, substituted or unsubstituted, of 1 to about 100 atoms, typically 1 to about 30 atoms, e.g., an optionally substituted alkylene, alkenylene, alkylyne, cycloalkyl, alkylcycloalkyl, alkylarylalkyl chain of 2 to 20 atoms optionally with 1-4 heteroatoms selected from —O—, —NH—, and —S—. Illustrative examples of L are a single or double covalent bond, C1-12 alkylene, substituted C1-12 alkylene, C1-12 alkenylene, substituted C1-12 alkenylene, C1-12 alkynylene, substituted C1-12 alkynylene, Xₙ-phenyl-Yₙ, or Xₙ-(phenyl)₂-Yₙ, wherein X and Y are independently C1-6 alkylene, substituted C1-6 alkylene, C1-6 alkenylene, substituted C1-6 alkenylene, C1-6 alkynylene, substituted C1-6 alkynylene, or S(O)₂.

Illustrative P3/P3′ groups include, without limitation:

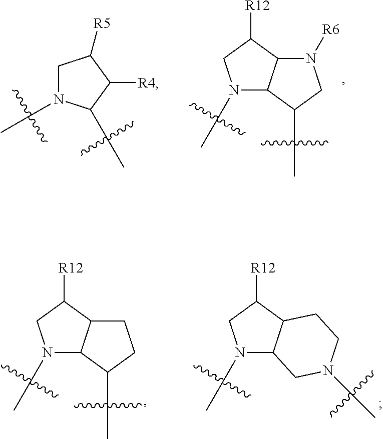

wherein R⁶ is —H, C1-6 alkyl, substituted C1-6 alkyl, C1-6 alkoxy, substituted C1-6 alkoxy, C1-6 alkylsulfonyl, arylsulfonyl, cycloalkyl, substituted cycloalkyl, heterocycloalkyl, substituted heterocycloalkyl, aryl, substituted aryl, heteroaryl, or substituted heteroaryl;
R⁴, R⁵, and R′² are, independently, —H, —OH, C1-6 alkyl, C1-6 heteroalkyl, C1-6 alkoxy, aryloxy, cycloalkyl, heterocycloalkyl, aryl, C1-6 alkyl aryl, or heteroaryl, or C1-6 alkyl heteroaryl, optionally substituted in each case except when R⁴ is —H or —OH.

As mentioned, in certain illustrative embodiments, the Smac mimetic used in the practice of the invention is bivalent.

Compound 15, i.e., birinapant, is an example of a specific Smac mimetic. Other illustrative examples are:

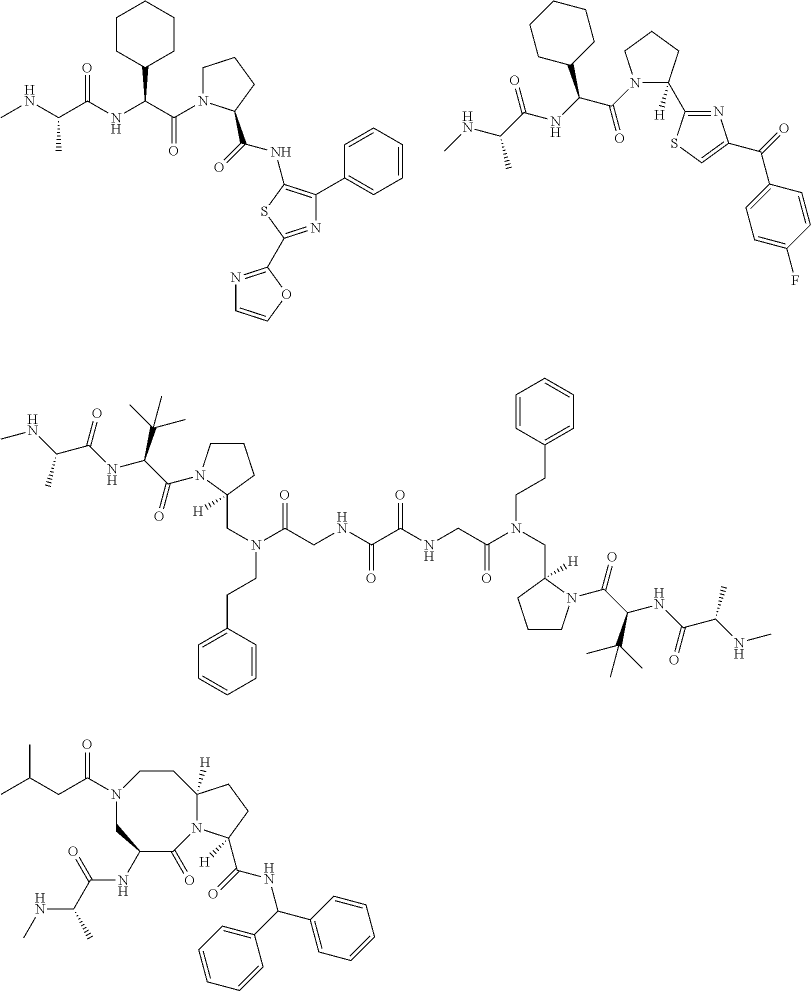

In certain illustrative embodiments, a selected Smac mimetic derepresses XIAP-mediated caspase-3 repression and/or degrades cIAP-1 not bound to TRAF2 (non TRAF2-bound, e.g., “cytoplasmic” cIAP-1 or “free” cIAP-1) as well as cIAP1 bound to TRAF2 and/or degrades cIAP-2 bound to TRAF2 but does not degrade cIAP-2 not bound to TRAF2 or weakly degrades cIAP-2 not bound to TRAF2 relative to degradation of cIAP-2 bound to TRAF2.

As mentioned above, this invention is applicable to treatment of patients suffering from pathogenic infection. Use of a SM in the treatment of intracellular infection is described in PCT/AU2014/050092, entitled “Method of Treating Intracellular Infection”. So, for example, patients with Hepatitis B Virus infection could have a liver biopsy after treatment to see if there was a change in the level of activated NF-κB. such as a change in the level of activated NF-κB relative to the level of unactivated NF-κB, (The biopsy would likely include non-infected liver cells, but these could be employed as a surrogate for NF-κB activation in target cells, i.e., in infected cells.) By way of further example, in the case of a patient suffering from tuberculosis, NF-κB activation can be determined in cells derived from sputum. By way of yet further example, in a patient suffering from HIV infection, cells that are infected are available in the circulation, and could be easily accessed and used for determining NF-κB activation directly in target cells.

In illustrative embodiments, this invention comprises a diagnostic kit for determining activation of NF-κB. Such kit may comprise various collecting and sampling devices and/or reagents including but not limited to liquid carriers for placement of cell samples that may be useful for all or some of the assay steps.

The parts of the kit of the invention can be packaged individually in vials or other appropriate means depending on the respective ingredient or in combination in suitable containers or multicontainer units. Manufacture of the kit can follow standard procedures which are known to the person skilled in the art. The kit may be used for methods for detecting expression of genes or polynucleotides in accordance with any one of the above-described methods of the invention, employing, for example, nucleic acid hybridization and/or amplification techniques such as those described herein before and in the examples.

As new techniques are developed for quantifying relative or absolute NF-κB activation, such techniques can be adapted for use in the present invention.

NF-κB translocation information, as described herein, may be stored in a computer readable form. Such a computer system typically comprises major subsystems such as a central processor, a system memory (typically RAM), an input/output (I/O) controller, an external device such as a display screen via a display adapter, serial ports, a keyboard, a fixed disk drive via a storage interface and optionally, a disk drive operative to receive a floppy disc, a CD or DVD, or any other data storage medium. Many other devices can be connected, such as a closed or open network interface.

The computer system may be linked to a network, comprising a plurality of computing devices linked via a data link, such as a cable, telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The computer system can comprise code for analyzing, i.e., interpreting, the results of analyses as described herein. Thus in an exemplary embodiment, the analytical results are provided to a computer where a central processor executes a computer program for determining a likelihood of response to treatment with a SM.

The invention also provides the use of a computer system, such as that described above, which comprises: (1) a computer including a computer processor; (2) a stored bit pattern encoding the results obtained by the analyses of the invention, which may be stored in the computer; (3) and, optionally, (4) a program for determining the likelihood of a therapeutic response.

A computer-based system for use in the methods described herein generally includes at least one computer processor (e.g., where the method is carried out in its entirety at a single site) or at least two networked computer processors (e.g., where data is to be input by a user (also referred to herein as a “client”) and transmitted to a remote site to a second computer processor for analysis, where the first and second computer processors are connected by a network, e.g., via an intranet or internet). The system can also include a user component(s) for input; and a reviewer component(s) for review of data, generated reports, and manual intervention. Additional components of the system can include a server component(s); and a database(s) for storing data (e.g., as in a database of report elements, e.g., interpretive report elements, or a relational database (RDB) which can include data input by the user and data output. The computer processors can be processors that are typically found in personal desktop computers (e.g., IBM, Dell, Apple), portable computers, mainframes, minicomputers, or other computing devices.

A networked client/server architecture can be selected as desired, and can be, for example, a classic two or three tier client server model. A relational database management system (RDMS), either as part of an application server component or as a separate component (RDB machine) provides the interface to the database.

In one example, the architecture is provided as a database-centric client/server architecture, in which the client application generally requests services from the application server which makes requests to the database (or the database server) to populate the report with the various report elements as required, particularly the interpretive report elements, especially the interpretation text and alerts. The server(s) (e.g., either as part of the application server machine or a separate RDB/relational database machine) responds to the client's requests.

The input client components can be complete, stand-alone personal computers offering a full range of power and features to run applications. The client component usually operates under any desired operating system and includes a communication element (e.g., a modem or other hardware for connecting to a network), one or more input devices (e.g., a keyboard, mouse, keypad, or other device used to transfer information or commands), a storage element (e.g., a hard drive or other computer-readable, computer-writable storage medium), and a display element (e.g., a monitor, television, LCD, LED, or other display device that conveys information to the user). The user enters input commands into the computer processor through an input device. Generally, the user interface is a graphical user interface (GUI) written for web browser applications.

The server component(s) can be a personal computer, a minicomputer, or a mainframe and offers data management, information sharing between clients, network administration and security. The application and any databases used can be on the same or different servers.

Other computing arrangements for the client and server(s), including processing on a single machine such as a mainframe, a collection of machines, or other suitable configuration are contemplated. In general, the client and server machines work together to accomplish the processing of the present invention.

Where used, the database(s) is usually connected to the database server component and can be any device which will hold data. For example, the database can be any magnetic or optical storing device for a computer (e.g., CD-ROM, internal hard drive, tape drive). The database can be located remote to the server component (with access via a network, modem, etc.) or locally to the server component.

Where used in the system and methods, the database can be a relational database that is organized and accessed according to relationships between data items. The relational database is generally composed of a plurality of tables (entities). The rows of a table represent records (collections of information about separate items) and the columns represent fields (particular attributes of a record). In its simplest conception, the relational database is a collection of data entries that “relate” to each other through at least one common field.

Additional workstations equipped with computers and printers may be used at point of service to enter data and, in some embodiments, generate appropriate reports, if desired. The computer(s) can have a shortcut (e.g., on the desktop) to launch the application to facilitate initiation of data entry, transmission, analysis, report receipt, etc. as desired.

The present invention also contemplates a computer-readable storage medium (e.g. CD-ROM, memory key, flash memory card, diskette, etc.) having stored thereon a program which, when executed in a computing environment, provides for implementation of algorithms to carry out all or a portion of the results of a response likelihood assessment as described herein. Where the computer-readable medium contains a complete program for carrying out the methods described herein, the program includes program instructions for collecting, analyzing and generating output, and generally includes computer readable code devices for interacting with a user as described herein, processing that data in conjunction with analytical information, and generating unique printed or electronic media for that user.

Where the storage medium provides a program that provides for implementation of a portion of the methods described herein (e.g., the user-side aspect of the methods (e.g., data input, report receipt capabilities, etc.)), the program provides for transmission of data input by the user (e.g., via the internet, via an intranet, etc.) to a computing environment at a remote site. Processing or completion of processing of the data is carried out at the remote site to generate a report. After review of the report, and completion of any needed manual intervention, to provide a complete report, the complete report is then transmitted back to the user as an electronic document or printed document (e.g., fax or mailed paper report). The storage medium containing a program according to the invention can be packaged with instructions (e.g., for program installation, use, etc.) recorded on a suitable substrate or a web address where such instructions may be obtained. The computer-readable storage medium can also be provided in combination with one or more reagents for carrying out response likelihood assessment (e.g., primers, probes, arrays, or other such kit components).

The invention further provides methods of generating a report based on the analyses of NF-κB activation in a patient suffering from a proliferative disorder. In general, such method can comprise the steps of determining information indicative of the activation of NF-κB and, optionally, of one or more biomarkers, in said tumor sample; and creating a report summarizing said information, with or without additional information.

In one aspect of the method, if the NF-κB activation data are indicative of possible responsiveness to treatment with a SM, said report includes an indication that the patient is a candidate for SM therapy. In another aspect of the method, if the data are related to non-responsiveness to treatment with a SM, said report includes a prediction that said subject has a low likelihood of response to treatment with such agent.

In one aspect, the report includes information to support a treatment recommendation for said patient. For example, the information can include a recommendation for adjuvant chemotherapy and/or neoadjuvant chemotherapy, a likelihood of chemotherapy benefit score, or other such data.

In another aspect the present disclosure provides reports for a patient containing a summary of the expression levels of the one or more biomarker genes, or their expression products, in a tumor sample obtained from said patient. In some embodiments, the report includes the likelihood of responsiveness.

Such report can further include one or more of: 1) information regarding the testing facility; 2) service provider information; 3) patient data; 4) sample data; 5) an interpretive report, which can include various information including: a) indication; b) test data, where test data can include a normalized level of one or more genes of interest, and 6) other features.

In some embodiments, the report further includes a recommendation for a treatment modality for said patient. In all aspects the report may include a classification of a subject into a risk group. In all aspects a report may include a prediction of the likelihood that said patient will respond positively to treatment with a pro-apoptotic agent.

In some embodiments, the report is in electronic form e.g., presented on an electronic display (e.g., computer monitor).

A person or entity who prepares a report (“report generator”) may also perform the likelihood assessment. The report generator may also perform one or more of sample gathering, sample processing, and data generation, e.g., the report generator may also perform one or more of: a) sample gathering; b) sample processing; c) measuring a level of NF-κB activation. Alternatively, an entity other than the report generator can perform one or more sample gathering, sample processing, and data generation.

For clarity, it should be noted that the term “user,” which is used interchangeably with “client,” is meant to refer to a person or entity to whom a report is transmitted, and may be the same person or entity who does one or more of the following: a) collects a sample; b) processes a sample; c) provides a sample or a processed sample; and d) generates data (e.g., level of a response indicator gene product(s); level of a reference gene product(s); normalized level of a response indicator gene product(s)) for use in the likelihood assessment. In some cases, the person(s) or entity(ies) who provides sample collection and/or sample processing and/or data generation, and the person who receives the results and/or report may be different persons, but are both referred to as “users” or “clients” herein to avoid confusion. In certain embodiments, e.g., where the methods are completely executed on a single computer, the user or client provides for data input and review of data output. A “user” can be a health professional (e.g., a clinician, a laboratory technician, a physician (e.g., an oncologist, surgeon, pathologist, etc.).

In embodiments where the user only executes a portion of the method, the individual who, after computerized data processing according to the methods of the invention, reviews data output (e.g., results prior to release to provide a complete report, a complete, or reviews an “incomplete” report and provides for manual intervention and completion of an interpretive report) is referred to herein as a “reviewer.” The reviewer may be located at a location remote to the user (e.g., at a service provided separate from a healthcare facility where a user may be located).

Where government regulations or other restrictions apply (e.g., requirements by health, malpractice, or liability insurance, or policy), results, whether generated wholly or partially electronically, are subjected to a quality control routine prior to release to the user.

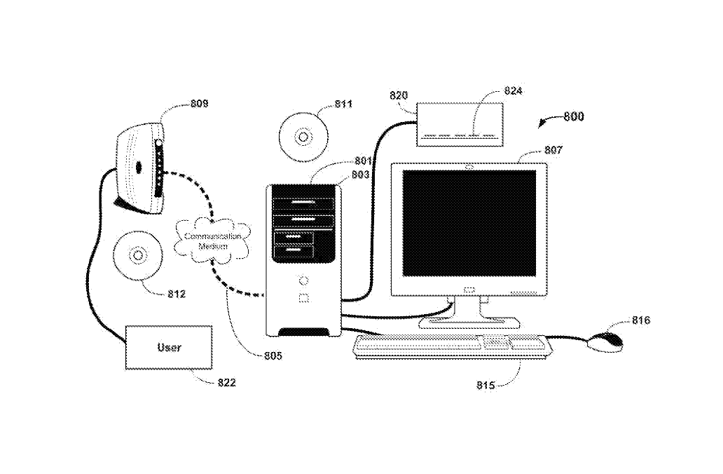

FIG. 1 is a block diagram showing a representative example logic device through which reviewing or analyzing data relating to the present invention can be achieved. Such data can be in relation to a disease, disorder or condition in an individual amenable to treatment with a pro-apoptotic agent, e.g., a Smac mimetic. FIG. 1 shows a computer system (or digital device) 800 connected to an apparatus 820 for use with the scanning sensing system 824 to, for example, produce a result. The computer system 800 may be understood as a logical apparatus that can read instructions from media 811 and/or network port 805, which can optionally be connected to server 809 having fixed media 812. The system shown in FIG. 1 includes CPU 801, disk drives 803, optional input devices such as keyboard 815 and/or mouse 816 and optional monitor 807. Data communication can be achieved through the indicated communication medium to a server 809 at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present invention can be transmitted over such networks or connections for reception and/or review by a party 822. The receiving party 822 can be but is not limited to a user, a scientist, a clinician, patient, a health care provider or a health care manager. In one embodiment, a computer-readable medium includes a medium suitable for transmission of a result of an analysis of a biological sample. The medium can include a result regarding a disease condition or state of a subject, wherein such a result is derived using the methods described herein.

In another related aspect of the present invention, the present disclosure concerns methods of preparing a personalized genomics profile for a patient by a) determining the normalized NF-κB activation levels and optionally other biomarkers as described in this specification, or their expression products, in a tumor sample obtained from said patient; and (b) creating a report summarizing the data.

For all aspects of the methods of the present disclosure, the determination of NF-κB activation levels may occur more than one time.

For all aspects of the methods of the present disclosure, the determination of levels may occur before the patient is subjected to any therapy following surgical resection.

For all aspects of the methods of the present disclosure, it is contemplated that administration of a pro-apoptotic agent may be as an adjunct to other therapy or as part of a combination therapy or both.

For all aspects of the methods of the present disclosure, the methods may further include the step of creating a report summarizing said likelihood.

The SM employed in the present invention can be administered to a patient either alone or as part of a pharmaceutical composition in a therapeutically effective amount. A variety of non-limiting methods for administering such compounds and related compositions to patients include orally, rectally, parenterally (intravenously, intramuscularly, or subcutaneously), intracisternally, intravaginally, intraperitoneally, intravesically, locally (powders, ointments, or drops), or as a buccal or nasal spray. In addition, the substance or compositions containing the active substance can be administered all at once, as for example, by a bolus injection, multiple times, such as by a series of tablets, or delivered substantially uniformly over a period of time, as for example, using transdermal delivery. It is also noted that the dose of the substances can be varied over time.

The compounds and pharmaceutical compositions employed in the present invention can be administered alone, or in combination with other pharmaceutically active substances. The other pharmaceutically active substances can be intended to treat the same disease or condition as the substance employed in the present invention or a different disease or condition. If the patient is to receive, or is receiving multiple pharmaceutically active substances, the substances can be administered simultaneously, or sequentially. For example, in the case of tablets, the active substances may be found in one tablet or in separate tablets, which can be administered at once or sequentially in any order. In addition, it should be recognized that the compositions may be different forms. For example, one or more substances may be delivered via a tablet, while another is administered via injection or orally as a syrup. All combinations, delivery methods and administration sequences are contemplated.

Pharmaceutical compositions to be used comprise a therapeutically effective amount of a Smac mimetic as described above, or a pharmaceutically acceptable salt or other form thereof together with one or more pharmaceutically acceptable excipients. The phrase “pharmaceutical composition” refers to a composition suitable for administration in medical or veterinary use. It should be appreciated that the determinations of proper dosage forms, dosage amounts, and routes of administration for a particular patient are within the level of ordinary skill in the pharmaceutical and medical arts.

Compositions suitable for parenteral administration, e.g., intravenous bolus or infusion, conveniently comprise a sterile aqueous preparation of such compound, which is preferably isotonic with the blood of the recipient. This aqueous preparation may be formulated according to known methods using suitable dispersing or wetting agents, emulsifying and suspending agents. Various antibacterial and antifungal agents, for example, parabens, chlorobutanol, phenol, and sorbic acid also may be included. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Prolonged absorption of the injectable pharmaceutical form can be brought about by the use of agents delaying absorption, for example, aluminum monostearate and gelatin. Carrier formulation suitable for subcutaneous, intravenous, intramuscular, etc. administrations can be found in Remington's Pharmaceutical Sciences, Mack Publishing Co., Easton, Pa.

Solid dosage forms for oral administration include capsules, tablets, pills, powders, and granules. In such solid dosage forms, the compound is admixed with at least one inert pharmaceutically acceptable excipient such as (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid dosage forms such as tablets, dragees, capsules, pills, and granules also can be prepared with coatings and shells, such as enteric coatings and others well known in the art. The solid dosage form also may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients. Such solid dosage forms may generally contain from 1% to 95% (w/w) of the active compound. In certain embodiments, the active compound ranges from 5% to 70% (w/w).

Liquid dosage forms for oral administration include pharmaceutically acceptable emulsions, solutions, suspensions, syrups, and elixirs. In addition to the Smac mimetic, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances. Besides such inert diluents, the composition can also include adjuvants, such as wetting agents, emulsifying and suspending agents, sweetening, flavoring, and perfuming agents.

The compounds and compositions of the present invention also may benefit from a variety of delivery systems, including time-released, delayed release or sustained release delivery systems. Such option may be particularly beneficial when the compounds and composition are used in conjunction with other treatment protocols as described in more detail below.

Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be desirable. Long-term release, as used herein, means that the implant is constructed and arranged to deliver therapeutic levels of the active compound for at least 30 days, and preferably 60 days. Long-term sustained release implants are well-known to those of ordinary skill in the art and include some of the release systems described above.

In an illustrative embodiment of the invention, a Smac mimetic is administered in a therapeutically effective amount alone or in combination with another agent, e.g., a Death Receptor Agonist such as TRAIL or a TRAIL-agonist antibody. Generally, doses of such Smac mimetics would be from about 0.01 mg/kg per day to 1000 mg/kg per day. It is expected that doses ranging from 50-500 mg/kg will be suitable, e.g., intravenously or orally, and in one or several administrations per day. The compounds of the present invention may also be used in combination with radiation therapy, hormone therapy, surgery and immunotherapy, which therapies are well known to those skilled in the art.

In general, routine experimentation in clinical trials will determine specific ranges for optimal therapeutic effect for a particular pro-apoptotic agent and each administrative protocol, and administration to specific patients will be adjusted to within effective and safe ranges depending on the patient condition and responsiveness to initial administrations. However, the ultimate administration protocol will be regulated according to the judgment of the attending clinician considering such factors as age, condition and size of the patient, the potency of the compound or composition, the duration of the treatment and the severity of the disease being treated. For example, a dosage regimen of a Smac mimetic can be an oral administration of from 1 mg to 2000 mg/day, preferably 1 to 1000 mg/day, more preferably 50 to 600 mg/day, in two to four (preferably two) divided doses, to reduce tumor growth. Intermittent therapy (e.g., one week out of three weeks or three out of four weeks) may also be used.

Suitable dosing regimens for a particular Smac mimetic are disclosed, e.g., in US patent application Ser. No. 13/751,959, filed Jan. 28, 2013.

In the event that a response in a subject is insufficient at the initial doses applied, higher doses (or effectively higher doses by a different, more localized delivery route) may be employed to the extent that the patient tolerance permits. Multiple doses per day may be used to achieve appropriate systemic levels of the Smac mimetic. Generally, a maximum dose is used, that is, the highest safe dose according to sound medical judgment, i.e., a MTD. Those of ordinary skill in the art will understand, however, that a patient may insist upon a lower dose or tolerable dose for medical reasons, psychological reasons or for any or a number of other reasons.

Thus, the pharmaceutical composition of the invention is a composition in which the active pharmaceutical ingredient, i.e., a SM such as birinapant, is pure enough, and the composition is otherwise suitable, for internal administration to a human or other mammal. It can be prepared in unit dose form, i.e., a form suitable for single administration to a subject such as by infusion. So, e.g., a pharmaceutical composition in intravenous unit dose form may comprise a vial or pre-filled syringe, or an infusion bag or device, each comprising a sufficient amount of birinapant to supply the desired dose (or a convenient fraction of such dose), as described hereinafter, such that the contents of one vial or syringe (or a small number of multiple vials, depending upon the fraction of dose in each) are administered at a time.

Administration can be repeated up to about 4 times per day over a period of time, if necessary to achieve a cumulative effective dose, e.g., a cumulative dose effective to produce tumor stasis or regression. A dosing regimen can be, e.g., daily, twice-weekly, or three times weekly (i.e., thrice weekly) intravenous injections, or, e.g., once weekly injections in cycles of three weeks on and one week off, or continuously, for as long as the treatment is effective, e.g., until disease progresses or the drug is not tolerated. The effective dose administered in each injection is an amount that is effective and tolerated.

An effective dose is one that over the course of therapy, which may be, e.g., 1 or more weeks, e.g., multiple courses of 3 weeks on/1 week off, results in treatment of the proliferative disorder, i.e., a decrease in the rate of disease progression, termination of disease progression, or regression or remission.

Birinapant has been found to be unexpectedly well tolerated. It can therefore, in general, be administered in doses that are higher than previously understood. In some embodiments of the invention, birinapant can, in general, be administered in doses that are generally higher than other synthetic small molecules that mimic the structure and IAP antagonist activity of the four N-terminal amino acids of Smac (i.e., other Smac mimetics). Other Smac mimetics have lower maximum tolerated doses (MTD) and have not shown meaningful clinical efficacy below such MTDs.

Doses employed in the practice of this invention can be effective in potentiating apoptosis of abnormally proliferating cells in a patient suffering a proliferative disorder or certain other disorders, e.g., certain autoimmune disorders. For example, birinapant can be administered intravenously, e.g., by infusion, at a dose of 1 to 80 mg/m² of patient body surface area (BSA) per day of treatment, e.g., 2 to 80, 2 to 65, 5 to 65, 10 to 65, 20 to 65, 30 to 65, 30 or >30 to 80, 30 or >30 to 65, 30 or >30 to 60, 30 or >30 to 55, or 30 or >30 to 50 mg/m², administered, e.g., by infusion over about 1 to about 120 minutes, e.g., about 30 minutes. The dose in most cases will be more than 5 mg/m². For example, the dose can be in the range 5 or >5 to 80, 5 or >5 to 60 mg/m². Current clinical studies employ about 5 mg/m² to about 50 mg/m², specifically, 5.6 to 47 mg/m². In two patients who received 63 mg/m², weekly/3 weeks on, /1 week off, birinapant was not well tolerated.

It will be understood that there are different formulae for calculating BSA. Most commonly used are the Mosteller formula (Mosteller RD. “Simplified calculation of body-surface area”. N Engl J Med 317:1098 (1987)) and the Dubois & Dubois formula (Du Bois & Du Bois, Arch Intern Med 17:863 (1916)). Doses recited herein are meant to apply to BSA calculated as per any such accepted methodologies notwithstanding that such different methodologies may result in slightly different BSA calculations, e.g., depending upon the number of decimal places used. It is generally sufficient to round off BSA calculations to 1 decimal place with allowance for a reasonable margin of error, e.g., 1.6 m² (+/−0.1) or 1.9 m² (+/−0.1). For purposes of this invention, BSA can also be estimated, e.g., using relevant population averages.

Doses recited herein as mg/m² BSA can, of course, be converted to mg/kg body weight. So, for example, assuming a given patient has a BSA of 1.6 m² and a body weight of 77 kg, a dose of 40 mg/m² is equal to a dose of 64 mg, i.e., about 0.8 mg/kg. By way of further example, using an average adult BSA of 1.7 m² and an average adult body weight of 70 kg, a dose of 40 mg/m² is equal to a dose of 68 mg, i.e., also about 0.8 mg/kg. Similarly, a dose range of >30 to 60 mg/m² equates to a dose range of >0.7 mg/kg to approximately 1.5 mg/kg, in such person of average BSA and weight.

It has also been discovered that birinapant has a long half-life in the patient and therefore can be administered less often than once per day. In general, birinapant can be administered once, twice or three times per week for one to four weeks (or longer). In some situations a treatment interval may be followed by a rest interval. A suitable rest interval includes but is not limited to one week. Such treatment cycle of one, two, three or four weeks “on” and one week “off” can be continued for as long as the drug shows effectiveness and is tolerated. It should be understood that the “on” weeks are consecutive weeks, i.e., two consecutive weeks on drug, three consecutive weeks on drug, and four consecutive weeks (or more) on drug.

An illustrative dosing regimen for birinapant is one ˜30 minute infusion/week for one to four weeks, e.g., once a week for 2 or 3 consecutive weeks, followed by a week off Specific illustrative dosing regimens include, without limitation, one administration by, e.g., intravenous infusion, of drug per week, in accordance with one of the following treatment cycles:
1) two weeks on/one week off, e.g., in combination with chemotherapies;
2) one week on/one week off, e.g., in patients with AML;
3) two weeks on/one week off, e.g., in patients with AML;
4) three weeks on/one week off, e.g., in patients with AML;
5) continuously (i.e., without a rest interval).

An illustrative dosing regimen for birinapant is one 30 minute infusion/week for 2 to 4 weeks, e.g., once a week for 2 or 3 consecutive weeks, followed by a week off. Such treatment cycle of two, three or four weeks on and one week off can be continued for as long as birinapant shows effectiveness and is tolerated.

In an alternative dosing regimen, birinapant is administered weekly, twice weekly, or three times per week, without a rest interval, i.e., continuously, for as long as birinapant shows effectiveness and is tolerated.

Typically, higher doses will be employed when birinapant is used in monotherapy, i.e., single agent therapy, then in combination therapy. Such monotherapy dose can be, e.g., about 40 to about 55 mg/m², or about 45 to about 50 mg/m², weekly for three weeks on/one week off or weekly continuously. An illustrative dosing regimen for birinapant in single agent therapy is 45 to 50 mg/m², e.g., 47 mg/m², weekly for three weeks on/one week off or weekly continuously.

When birinapant is used in combination therapy, the dose can be, e.g., about 5 to about 50 mg/m², or about 5 to about 40 mg/m², weekly for three weeks on/one week off or weekly continuously. An illustrative dosing regimen for birinapant in combination therapy is about 5 to about 35 mg/m², weekly for three weeks on/one week off or weekly continuously.

In patients in whom birinapant is less well tolerated, lower doses can be administered more frequently. For example, in AML patients, birinapant can be administered in single agent therapy at about 15 to about 20 mg/m², e.g., 17 mg/m², twice/week (e.g., Mondays and Thursdays, Tuesdays and Fridays, etc.) or 17 mg mg/m², thrice/week (e.g., Mondays, Wednesdays, Fridays). three weeks on/one week off or continuously.

A SM, e.g., birinapant, can be administered in accordance with an ascending dose protocol. An ascending dose protocol is one in which the drug is initially administered at a dose lower than the target dose and is administered at increasingly higher doses in subsequent administrations until a target dose is reached. The initial dose is a dose that is unlikely to result in an adverse event and may be sub-therapeutic. The target dose is the dose that has been determined through clinical studies to be a safe and effective dose. Dose escalation is typically carried out by increasing the dose incrementally over 3 or more administrations until the target dose is achieved.

By way of illustration only, in one clinical study, irinotecan at 350 mg/m² q3 weeks was administered intravenously with birinapant weekly (2 of 3 weeks). For Cycle 1, the dose of birinapant was increased from 5.6 mg/m² on Day 1 to 11 mg/m² on Day 8). For Cycle 2 and ongoing treatment, birinapant was 22 mg/m² or 35 mg/m².

The method of the invention can be used to treat a subject suffering from cancer, an autoimmune disease or another disorder where a defect in apoptosis is implicated. In connection with such treatments, the patient can be treated prophylactically, acutely, or chronically using compounds and compositions of the present invention, depending on the nature of the disease. Typically, the host or subject in each of these methods is human, although other mammals may also benefit from the administration of a compound of the present invention.

As described in U.S. Pat. No. 7,244,851, Smac mimetics can be used for the treatment of cancer types that fail to undergo adequate apoptosis. Thus, compounds used on the method of the present invention can be used to provide a therapeutic approach to the treatment of many kinds of solid tumors, including but not limited to carcinomas, sarcomas including Kaposi's sarcoma, erythroblastoma, glioblastoma, meningioma, astrocytoma, melanoma and myoblastoma. Treatment or prevention of non-solid tumor cancers such as leukemia is also contemplated by this invention. Indications may include, but are not limited to brain cancers, skin cancers, bladder cancers, ovarian cancers, breast cancers, gastric cancers, pancreatic cancers, colon cancers, blood cancers, lung cancers and bone cancers. Examples of such cancer types include neuroblastoma, intestine carcinoma such as rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testes carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngeoma, osteosarcoma, chondrosarcoma, myosarcoma, liposarcoma, fibrosarcoma, Ewing sarcoma and plasmocytoma.

SMs selected for use in accordance with the present invention will be active for treating human malignancies including, but not limited to, such human malignancies where cIAP1 and cIAP2 are over-expressed (e.g., lung cancers, see Dai et al, Hu. Molec. Genetics, 2003 v 12 pp. 791-801; leukemias (multiple references), and other cancers (Tamm et al, Clin Cancer Res, 2000, v 6, 1796-1803). Such pro-apoptotic agents will be active in disorders that may be driven by inflammatory cytokines such as TNFαplaying a pro-survival role (for example, there is a well defined role for TNFαacting as a survival factor in ovarian carcinoma, similarly for gastric cancers (see Kulbe, et al, Cancer Res 2007, 67, 585-592).

In addition to apoptosis defects found in tumors, defects in the ability to eliminate self-reactive cells of the immune system due to apoptosis resistance are considered to play a key role in the pathogenesis of autoimmune diseases. Autoimmune diseases are characterized in that the cells of the immune system produce antibodies against its own organs and molecules or directly attack tissues resulting in the destruction of the latter. A failure of those self-reactive cells to undergo apoptosis leads to the manifestation of the disease. Defects in apoptosis regulation have been identified in autoimmune diseases such as systemic lupus erthematosus or rheumatoid arthritis.

Examples of such autoimmune diseases include collagen diseases such as rheumatoid arthritis, systemic lupus erythematosus, Sharp's syndrome, CREST syndrome (calcinosis, Raynaud's syndrome, esophageal dysmotility, telangiectasia), dermatomyositis, vasculitis (Morbus Wegener's) and Sjögren's syndrome, renal diseases such as Goodpasture's syndrome, rapidly-progressing glomerulonephritis and membrano-proliferative glomerulonephritis type II, endocrine diseases such as type-I diabetes, autoimmune polyendocrinopathy-candidiasis-ectodermal dystrophy (APECED), autoimmune parathyroidism, pernicious anemia, gonad insufficiency, idiopathic Morbus Addison's, hyperthyreosis, Hashimoto's thyroiditis and primary myxedema, skin diseases such as pemphigus vulgaris, bullous pemphigoid, herpes gestationis, epidermolysis bullosa and erythema multiforme major, liver diseases such as primary biliary cirrhosis, autoimmune cholangitis, autoimmune hepatitis type-1, autoimmune hepatitis type-2, primary sclerosing cholangitis, neuronal diseases such as multiple sclerosis, myasthenia gravis, myasthenic Lambert-Eaton syndrome, acquired neuromyotony, Guillain-Barré syndrome (Müller-Fischer syndrome), stiff-man syndrome, cerebellar degeneration, ataxia, opsoklonus, sensoric neuropathy and achalasia, blood diseases such as autoimmune hemolytic anemia, idiopathic thrombocytopenic purpura (Morbus Werlhof), infectious diseases with associated autoimmune reactions such as AIDS, Malaria and Chagas disease.

The present invention also is directed to the use of the compounds and compositions as a chemopotentiating agent with other treatment approaches. The term “chemopotentiating agent” refers to an agent that acts to increase the sensitivity of an organism, tissue, or cell to a chemical compound, or treatment namely “chemotherapeutic agents” or “chemo drugs” or to radiation treatment. Thus, compounds and compositions of the present invention can be used for inhibiting tumor growth in vivo by administering them in combination with a biologic or chemotherapeutic agent or by using them in combination with chemoradiation. In these applications, the administration of the compounds and compositions of the present invention may occur prior to, and with sufficient time, to cause sensitization of the site to be treated. Alternatively, the methods and compositions of the present invention may be used contemporaneously with radiation and/or additional anti-cancer chemical agents (infra) Such systems can avoid repeated administrations of the methods and compositions of the present invention, increasing convenience to the subject and the physician, and may be particularly suitable for certain methods and compositions of the present invention.

Biological and chemotherapeutics/anti-neoplastic agents and radiation induce apoptosis by activating the extrinsic or intrinsic apoptotic pathways, and, since the compounds and compositons of the present invention relieve antagonists of apoptotic proteins (IAPs) and, thus, remove the block in apoptosis, the combination of chemotherapeutics/anti-neoplastic agents and radiation with the compounds and compositons of the present invention should work synergistically to facilitate apoptosis.

A combination of a pro-apoptotic agent, e.g., a Smac mimetic and a chemotherapeutic/antineoplastic agent and/or radiation therapy of any type that activates the intrinsic pathway may provide a more effective approach to destroying tumor cells. Smac mimetics interact with IAP's, such as XIAP, cIAP1, cIAP2, ML-IAP, etc., and block the IAP mediated inhibition of apoptosis while chemotherapeutics/antineoplastic agents and/or radiation therapy kills actively dividing cells by activating the intrinsic apoptotic pathway leading to apoptosis and cell death. As is described in more detail below, embodiments of the invention contemplate use of combinations of a pro-apoptotic agent, e.g., a Smac mimetic, and a chemotherapeutic/anti-neoplastic agent and/or radiation which provide a synergistic action against unwanted cell proliferation. This synergistic action between a Smac mimetic and a chemotherapeutic/anti-neoplastic agent and/or radiation therapy can improve the efficiency of the chemotherapeutic/anti-neoplastic agent and/or radiation therapies. This will allow for an increase in the effectiveness of current chemotherapeutic/anti-neoplastic agents or radiation treatments allowing the dose of the chemotherapeutic/anti-neoplastic agent to be lowered, therein providing both a more effective dosing schedule as well as use of a more tolerable dose of chemotherapeutic/anti-neoplastic agent and/or radiation.

In an embodiment of the present invention, the patient is treated by administering a Smac mimetic at a time the patient is subject to concurrent or antecedent radiation, surgery, hormone therapy, immunotherapy, or chemotherapy for treatment of a neoproliferative pathology of a tumor such as, but not limited to, bladder cancer, breast cancer, prostate cancer, lung cancer, pancreatic cancer, gastric cancer, colon cancer, ovarian cancer, renal cancer, hepatoma, melanoma, lymphoma, sarcoma, and combinations thereof.

In another embodiment of the present invention, a pro-apoptic agent can be administered can be administered to a patient having the appropriate level of NFKB activation in combination with a chemotherapeutic and/or for use in combination with radiotherapy, immunotherapy, and/or photodynamic therapy, promoting apoptosis and enhancing the effectiveness of the chemotherapeutic, radiotherapy, immunotherapy, and/or photodynamic therapy.

Chemotherapeutic agents include but are not limited to the chemotherapeutic agents described in “Modern Pharmacology with Clinical Applications”, Sixth Edition, Craig & Stitzel, Chpt. 56, pg 639-656 (2004). The chemotherapeutic agent can be, but is not limited to, alkylating agents, antimetabolites, anti-tumor antibiotics, plant-derived products such as taxanes, enzymes, hormonal agents, miscellaneous agents such as cisplatin, monoclonal antibodies, glucocorticoids, mitotic inhibitors, topoisomerase I inhibitors, topoisomerase II inhibitors, immunomodulating and other biological agents such as interferons, cellular growth factors, cytokines, and nonsteroidal anti-inflammatory compounds, cellular growth factors and kinase inhibitors. Other suitable classifications for chemotherapeutic agents include mitotic inhibitors and nonsteroidal anti-estrogenic analogs.

Specific examples of suitable biological and chemotherapeutic agents include, but are not limited to, cisplatin, carmustine (BCNU), 5-fluorouracil (5-FU), cytarabine (Ara-C), gemcitabine, methotrexate, daunorubicin, doxorubicin, dexamethasone, topotecan, etoposide, paclitaxel, vincristine, tamoxifen, TNF-alpha, TRAIL, interferon (in both its alpha and beta forms), thalidomide, and melphalan. Other specific examples of suitable chemotherapeutic agents include nitrogen mustards such as cyclophosphamide, alkyl sulfonates, nitrosoureas, ethylenimines, triazenes, folate antagonists, purine analogs, pyrimidine analogs, anthracyclines, bleomycins, mitomycins, dactinomycins, plicamycin, vinca alkaloids, epipodophyllotoxins, taxanes, glucocorticoids, L-asparaginase, estrogens, androgens, progestins, luteinizing hormones, octreotide actetate, hydroxyurea, procarbazine, mitotane, hexamethylmelamine, carboplatin, mitoxantrone, monoclonal antibodies, levamisole, interferons, interleukins, filgrastim and sargramostim. Chemotherapeutic compositions also comprise other members, i.e., other than TRAIL, of the TNF superfamily of compounds.

Another embodiment of the present invention relates to the use of a pro-apoptotic agent in a patient selected on the basis of his or her gene expression signature, as described herein, in combination with topoismerase inhibitors to potentiate their apoptotic inducing effect. Topoisomerase inhibitors inhibit DNA replication and repair, thereby promoting apoptosis and have been used as chemothemotherapeutic agents. Topoisomerase inhibitors promote DNA damage by inhibiting the enzymes that are required in the DNA repair process. Therefore, export of Smac from the mitochondria into the cell cytosol is provoked by the DNA damage caused by topoisomerase inhibitors. Topoisomerase inhibitors of both the Type I class (camptothecin, topotecan, SN-38 (irinotecan active metabolite)) and the Type II class (etoposide) are expected to show potent synergy with compounds of the present invention. Further examples of topoisomerase inhibiting agents that may be used include, but are not limited to, irinotecan, topotecan, etoposide, amsacrine, exatecan, gimatecan, etc. Other topoisomerase inhibitors include, for example, Aclacinomycin A, camptothecin, daunorubicin, doxorubicin, ellipticine, epirubicin, and mitaxantrone.

In another embodiment of the invention, the chemotherapeutic/anti-neoplastic agent for use in combination therapy in the present invention may be a platinum containing compound. In one embodiment of the invention, the platinum containing compound is cisplatin. Cisplatin can synergize with a compound of the present invention and potentiate the inhibition of an IAP, such as but not limited to XIAP, cIAP1, c-IAP2, ML-IAP, etc. In another embodiment a platinum containing compound is carboplatin. Carboplatin can synergize with a compound of the present invention and potentiate the inhibition of an IAP, including, but not limited to, XIAP, cIAP1, c-IAP2, ML-IAP, etc. In another embodiment a platinum containing compound is oxaliplatin. The oxaliplatin can synergize with a compound of the present invention and potentiate the inhibition of an IAP, including, but not limited to, XIAP, cIAP1, cIAP2, ML-IAP, etc.

Platinum chemotherapy drugs belong to a general group of DNA modifying agents. DNA modifying agents may be any highly reactive chemical compound that bonds with various nucleophilic groups in nucleic acids and proteins and cause mutagenic, carcinogenic, or cytotoxic effects. DNA modifying agents work by different mechanisms, disruption of DNA function and cell death; DNA damage/the formation of cross-bridges or bonds between atoms in the DNA; and induction of mispairing of the nucleotides leading to mutations, to achieve the same end result. Three non-limiting examples of a platinum containing DNA modifying agents are cisplatin, carboplatin and oxaliplatin.

Cisplatin is believed to kill cancer cells by binding to DNA and interfering with its repair mechanism, eventually leading to cell death. Carboplatin and oxaliplatin are cisplatin derivatives that share the same mechanism of action. Highly reactive platinum complexes are formed intracellularly and inhibit DNA synthesis by covalently binding DNA molecules to form intrastrand and interstrand DNA crosslinks.

Non-steroidal anti-inflammatory drugs (NSAIDs) have been shown to induce apoptosis in colorectal cancer cells. NSAIDs appear to induce apoptosis via the release of Smac from the mitochondria (PNAS, Nov. 30, 2004, vol. 101:16897-16902). Therefore, the use of NSAIDs in combination with the compounds and compositions of the present invention would be expected to increase the activity of each drug over the activity of either drug independently.

Many naturally occurring compounds isolated from bacterial, plant, and animals can display potent and selective biological activity in humans including anticancer and antineoplastic activities. In fact, many natural products, or semi-synthetic derivatives thereof, which possess anticancer activity, are already commonly used as therapeutic agents; these include paclitaxel, etoposide, vincristine, and camptothecin amongst others. Additionally, there are many other classes of natural products such as the indolocarbazoles and epothilones that are undergoing clinical evaluation as anticancer agents. A reoccurring structural motif in many natural products is the attachment of one or more sugar residues onto an aglycone core structure. In some instances, the sugar portion of the natural product is critical for making discrete protein-ligand interactions at its site of action (i.e., pharmacodynamics) and removal of the sugar residue results in significant reductions in biological activity. In other cases, the sugar moiety or moieties are important for modulating the physical and pharmacokinetic properties of the molecule. Rebeccamycin and staurosporine are representative of the sugar-linked indolocarbazole family of anticancer natural products with demonstrated anti-kinase and anti-topoisomerase activity.

Taxanes are anti-mitotic, mitotic inhibitors or microtubule polymerization agents. Taxanes are characterized as compounds that promote assembly of microtubules by inhibiting tubulin depolymerization, thereby blocking cell cycle progression through centrosomal impairment, induction of abnormal spindles and suppression of spindle microtubule dynamics. Taxanes include but are not limited to, docetaxel and paclitaxel. The unique mechanism of action of taxane is in contrast to other microtubule poisons, such as Vinca alkaloids, colchicine, and cryptophycines, which inhibit tubulin polymerization. Microtubules are highly dynamic cellular polymers made of alpha-beta-tubulin and associated proteins that play key roles during mitosis by participating in the organization and function of the spindle, assuring the integrity of the segregated DNA. Therefore, they represent an effective target for cancer therapy.

Yet another embodiment of the present invention employs the therapeutic combination or the therapeutic use in combination of a Smac mimetic with TRAIL or other chemical or biological agents which bind to and activate the TRAIL receptor(s). TRAIL has received considerable attention recently because of the finding that many cancer cell types are sensitive to TRAIL-induced apoptosis, while most normal cells appear to be resistant to this action of TRAIL. TRAIL-resistant cells may arise by a variety of different mechanisms including loss of the receptor, presence of decoy receptors, or overexpression of FLIP which competes for zymogen caspase-8 binding during DISC formation. In TRAIL resistance, a compound or composition of the present invention may increase tumor cell sensitivity to TRAIL leading to enhanced cell death, the clinical correlations of which are expected to be increased apoptotic activity in TRAIL resistant tumors, improved clinical response, increased response duration, and ultimately, enhanced patient survival rate. In support of this, reduction in XIAP levels by in vitro antisense treatment has been shown to cause sensitization of resistant melanoma cells and renal carcinoma cells to TRAIL (Chawla-Sarkar, et al., 2004). SMs bind to IAPs and inhibit their interaction with caspases, therein potentiating TRAIL-induced apoptosis.

The present invention also can be used to augment radiation therapy (or radiotherapy), i.e., the medical use of ionizing radiation as part of cancer treatment to control malignant cells. Although radiotherapy is often used as part of curative therapy, it is occasionally used as a palliative treatment, where cure is not possible and the aim is for symptomatic relief. Radiotherapy is commonly used for the treatment of tumors. It may be used as the primary therapy. It is also common to combine radiotherapy with surgery and/or chemotherapy. The most common tumors treated with radiotherapy are breast cancer, prostate cancer, rectal cancer, head & neck cancers, gynecological tumors, bladder cancer and lymphoma. Radiation therapy is commonly applied just to the localized area involved with the tumor. Often the radiation fields also include the draining lymph nodes. It is possible but uncommon to give radiotherapy to the whole body, or entire skin surface. Radiation therapy is usually given daily for up to 35-38 fractions (a daily dose is a fraction). These small frequent doses allow healthy cells time to grow back, repairing damage inflicted by the radiation. Three main divisions of radiotherapy are external beam radiotherapy or teletherapy, brachytherapy or sealed source radiotherapy and unsealed source radiotherapy, which are all suitable examples of treatment protocol in the present invention. The differences relate to the position of the radiation source; external is outside the body, while sealed and unsealed source radiotherapy has radioactive material delivered internally. Brachytherapy sealed sources are usually extracted later, while unsealed sources are injected into the body.

As mentioned above, an aspect of this invention is informing and educating patients, healthcare workers, and insurers about the relationship between NFKB activation and resistance to SM therapy so that a better informed decision can be made about whether or not to treat (or pay for) SM therapy for a given patient or population of patients. Such informing/educating can be accomplished in any of a number of ways include by advertising, marketing, seminars, continuing medical education, promotional advertising, etc. One useful way comprises including relevant information in the prescribing information, i.e., the “label,” that is approved for a given SM. The label content can be “soft”, e.g., merely providing data showing correlation between lack of NF-κB activation and SM-resistance, or “hard”, e.g., limiting the approved indication to treatment of patients in whose cancerous cells NF-κB have been shown to be activated at least to a certain level. In between the “soft” and “hard” content are multiple variations, e.g., recommendations, advice, and suggestions.

Explanations of mechanisms of action herein are intended to facilitate understanding of the invention but are not meant to be limiting. It is to be understood that the examples and embodiments described herein are for illustrative purposes only and that various modifications or changes in light thereof will be suggested to persons skilled in the art and are to be included within the spirit and purview of this application and the scope of the appended claims. All references cited hereinabove are incorporated herein by reference as though fully set forth.

1. A method of monitoring the response of a patient undergoing treatment with a SM, said method comprising monitoring levels of NF-κB activation in patient samples of target cells over time during a course of SM treatment, whereby decreasing levels of activated NF-κB in said cells is an indicator that the SM is having the desired pharmacologic effect.

2. The method of claim 1 wherein the patient is undergoing treatment with a SM in order to treat a cancer and wherein the target cells are abnormally proliferating cells.

3. The method of claim 2 wherein the cancer is MDS/AML.

4. The method of claim 2 wherein patient samples of targetted cells are collected and analyzed for NF-κB activation by imaging flow cytometry and measurement of relative amounts of cytoplasmic (unactivated) NF-κB and of nuclear (activated) NF-κB.

5. The method of claim 4 wherein levels of NF-κB activation are measured by calculating a similarity score that reflects the amount of cytoplasmic NF-κB relative to the amount of nuclear NF-κB.

6. The method of claim 4 wherein the SM is a peptidomimetic of the four N-terminal amino acids of Smac.

7. The method of claim 6 wherein the SM is birinapant.

8. The method of claim 7 wherein the cancer is MDS/AML.

9. A method of treating a patient for whom SM therapy is indicated, said method comprising:
(a) internally administering a SM to the patient during at least one treatment cycle,
(b) monitoring the response of the patient to the SM during each treatment cycle by monitoring levels of NF-κB activation in patient samples of targeted cells over time, whereby decreasing levels of activated NF-κB in said cells is an indicator that the SM is having the desired pharmacologic effect, and
(c) (i) discontinuing the SM treatment following completion of the at least one treatment cycle if the level of activated NF-κB relative to the level of unactivated NF-κB in target cells does not decrease while the patient is being treated with the SM or (c)(ii) continuing the SM treatment and the NF-κB monitoring for at least one additional treatment cycle following completion of the at least one treatment cycle if the level of activated NF-κB relative to the level of unactivated NF-κB in target cells decreases while the patient is being treated with the SM.

10. The method of claim 9 wherein SM therapy is indicated for the treatment of a cancer and wherein a treatment cycle comprises at least one administration of the SM per week for at least one week, optionally followed by a one week rest interval.

11. The method of claim 10 wherein the patient is treated with the SM by administering the SM to the patient one, two, or three times/week in accordance with one of the following treatment cycles:
two weeks on/one week off
one week on/one week off
two weeks on/one week off
three weeks on/one week off
no rest interval.

12. The method of claim 11 wherein the patient is undergoing treatment with a SM in order to treat a cancer and wherein the targetted cells are abnormally proliferating cells.

13. The method of claim 12 wherein the cancer is MDS/AML.

14. The method of claim 12 wherein patient samples of targetted cells are collected and analyzed for NF-κB activation by imaging flow cytometry and measurement of relative amounts of cytoplasmic (unactivated) NF-κB and of nuclear (activated) NF-κB.

15. The method of claim 14 wherein levels of NF-κB activation are measured by calculating a similarity score that reflects the amount of cytoplasmic NF-κB relative to the amount of nuclear NF-κB.

16. The method of claim 14 wherein the SM is a peptidomimetic of the four N-terminal amino acids of Smac.

17. The method of claim 16 wherein the SM has the structure:
[P1-P2-P3-P4] (Formula I)
or
[P1-P2-P3-P4]-L-[P1′-P2′-P3′-P4′] (Formula II)
wherein P1-P2-P3- and P1′-P2′-P3′- are amino acid replacements of, respectively, the N-terminal Ala-Val-Pro- of mature Smac and P4 and P4′ are amino acid replacements of the fourth N-terminal amino acid, Phe, Tyr, Ile, or Val, and L is a linking group or bond covalently linking [P1-P2-P3-P4] to [P1′-P2′-P3′-P4′].

18. The method of claim 17 wherein the SM has the structure of Formula II and wherein:
P1 and P1′ are NHR¹—CHR²—C(O)—;
P2 and P2′ are —NH—CHR³—C(O)—;
P3 and P3′ are independently pyrrolidine, pyrrolidine fused to a cycloalkyl, or pyrrolidine fused to a heterocycloalkyl having a—N— heteroatom, optionally substituted in each case, and wherein the pyrrolidine of P3/P3′ is bound to P2/P2′ by an amide bond;
P4 and P4′ are -M-Qₚ-R⁷;
each R¹ is independently —H or —CH3;
each R² is independently —CH3, —CH2CH3 or —CH2OH;
each R³ is independently C2-6 alkyl, C2-6 alkoxy, C3-C6 cycloalkyl or heterocycloalkyl, or C6-C8 aryl or heteroaryl, optionally substituted in each case;
each M is independently a covalent bond, C1-6 alkylene, or a substituted C1-C6 alkylene;
each Q is independently a covalent bond, C1-6 alkylene, substituted C1-C6 alkylene, —O— or —NR⁸—;
each P is independently 0 or 1;
each R⁷ is independently cycloalkyl, cycloalkylaryl, alkylaryl, alkylheteroaryl, aryl or heteroaryl, optionally substituted in each case;
each R⁸ is independently —H or C1-6 alkyl.

19. The method of claim 17 wherein the SM is selected from: 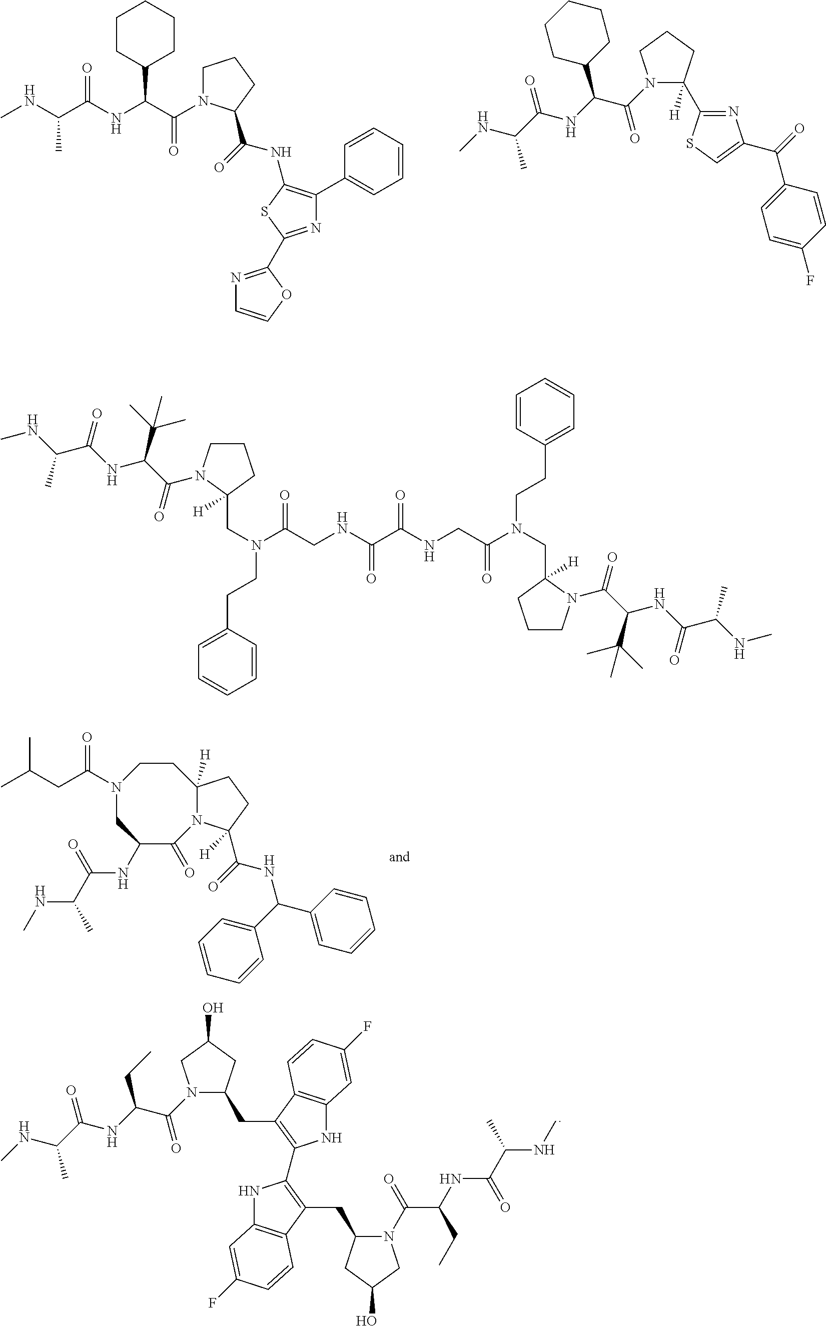
is birinapant.

20. The method of claim 17 wherein the cancer is MDS/AML.

21. A method of marketing a drug product comprising a Smac mimetic (“SM”) for the treatment of a disorder for which treatment with a SM is indicated, said method comprising informing patients, physicians or other healthcare providers, or insurers that the SM is unlikely to be effective in treating patients whose abnormally proliferating cells have an undetectable or low basal level of NF-κB activation.

22. A method of monitoring response of a patient suffering from a proliferative disorder to treatment with a SM, said method comprising monitoring levels of NF-κB activation in samples of the patient's abnormally proliferating cells over time, whereby decreasing levels of activated NF-κB in said cells is an indicator that the disorder is responding to the treatment.

23. A method of treating a patient suffering from a disorder for which treatment with a SM is indicated, said method comprising measuring the level of activated NF-κB in the patient's abnormally proliferating cells and:
if NF-κB is activated, e.g., if the level of activated NF-κB is high such as when p65 is present in the nucleus as measured, e.g. by imaging flow cytometry, then treating the patient by internally administering an effective amount of a SM; or,
if NF-κB is activated, e.g., if the level of activated NF-κB is low such as when p65 is not detected in the nucleus as measured, e.g. by imaging flow cytometry, then treating the patient with an alternative therapy.

24. A method of predicting the likelihood of efficacy of treatment of a patient suffering from a disorder for which treatment with a SM is indicated with a SM, said method comprising measuring the basal activity level of NF-κB in the patient's abnormally proliferating cells whereby if said basal activity level is low, then the patient is unlikely to respond to treatment with a SM.

25. A method of predicting the likelihood of efficacy of treatment of a patient suffering from a disorder for which treatment with a SM is indicated with a SM, said method comprising:
(i) obtaining a sample of abnormally proliferating cells associated with the disorder;
(ii) having the basal level of NF-κB activity in the cells measured;
(iii) receiving a measure of the basal level of NF-κB activity in the cells;
wherein if said basal activity level is low, then the patient is unlikely to respond to treatment with a SM.

26. A method of treating a patient suffering a disorder for which treatment with a SM is indicated that comprises:
(i) determining a basal level of NF-κB activity that correlates with a Treatment Threshold for a SM;
(ii) determining the basal level of NF-κB activity in abnormally proliferating cells from the patient;
(iii) if the basal level of NF-κB activity is at or greater than the Treatment Threshold, then internally administering to the patient an effective amount of the SM and if it is below the Treatment Threshold, then treating the patient with an alternative therapy.

27. A method of treating a patient suffering a disorder for which treatment with a SM is indicated that comprises:
(i) determining or otherwise receiving a measure of the basal level of NF-κB activity in the patient's abnormally proliferating cells, either at the time of treatment or from a previously obtained sample of abnormally proliferating cells;
(ii) if the level of NF-κB is above normal, then treating the patient by internally administering an effective amount of a SM; or,
if the level of NF-κB is normal or below normal, then treating the patient with an alternative therapy.

28. A method of stratifying patients suffering from a disorder for which treatment with a SM is indicated into at least two categories: (a) Likely Responders and (b) Likely Non-responders, said method comprising:
(i) determining or otherwise receiving a measure of the basal level of NF-κB activity in the patient's abnormally proliferating cells, either at the time of treatment or from a previously obtained sample of the patient's abnormally proliferating cells; or,
(ii) if said basal activity level is high, then assigning that patient to the Likely Responders category and if said basal activity level is low, then assigning that patient to the Likely Non-Responders category.

29. A method of treating a patient suffering a disorder for which treatment with a SM is indicated that comprises:
(i) obtaining a sample of abnormally proliferating cells associated with the disorder;
(ii) having the basal level of NF-κB activity in said cells measured;
(iii) receiving the measure of basal level of NF-κB activity in said cells,
wherein the basal level of NF-κB activity in said cells is directly related to the likelihood of efficacy of the treatment;
(iv) internally administering to the patient an effective amount of a SM if the basal level of NF-κB activity in said cells indicates a likelihood of efficacy of the treatment.

30. The method of any of claims 21-29 further defined by one or more of the following limitations:
the SM is birinapant;
the disorder for which treatment with a SM is indicated is a cancer;
the proliferative disorder is a NF-κB-dependent cancer;
the proliferative disorder is a leukemia;
the proliferative disorder is a myelodysplastic syndrome (MDS) or acute myeloid leukemia (AML);
a low basal level of NF-κB activation is normal or below normal level of NF-κB activation;
treatment with a SM is in combination therapy with a chemotherapeutic (including without limitation, a biological) agent and/or radiation
NF-κB activation is determined by assaying for a phosphorylated NF-κB protein in the nucleus of abnormally proliferating cells;
NF-κB activation is determined by assaying for RelA (p65) in the nucleus of abnormally proliferating cells;
NF-κB activation is determined by assaying for a NF-κB protein, e.g., RelA, in the nucleus of abnormally proliferating cells by use of imaging flow cytometery and NF-κB is determined to be activated if NF-κB protein, e.g., RelA, is detected in the nucleus in amounts that are higher than amounts in normal cells and/or NF-κB is determined not to be activated if NF-κB protein, e.g., RelA, is not detected in the nucleus or is detected in the nucleus in amounts that are normal or below normal.

31. The method of any of claims 21-29 applied to the treatment of a cancer or pre-cancerous condition.

32. The method of any of claims 21 to 29 applied to the treatment of MDS/AML.

33. The method of any of claims 21 to 29 applied to the treatment of an autoimmune disorder.

34. The method of any of claims 21 to 29 applied to the treatment of infection.

35. A computer system that comprises: (1) a computer processor; (2) a stored bit pattern encoding the basal NF-κB activity level in a sample of abnormally proliferating cells from a patient suffering from a disorder for which treatment with a SM is indicated.

36. A computer system that comprises: (1) a computer including a computer processor; (2) a stored bit pattern encoding information relating to the basal NF-κB activity level in a sample of abnormally proliferating cells from a patient suffering from a disorder for which treatment with a SM is indicated, which may be stored in the computer; (3) and a program for quantifying in absolute or relative terms the level of NF-κB activation in said cells.
